(19) United States
(12) Patent Application Publication
(10) Pub. No.: US 2026/0090717 A1
(43) Pub. Date: Apr. 2, 2026

(54) Ophthalmic System

(71) Applicant: Carl Zeiss Meditec, Inc., Dublin, CA (US)

(72) Inventors: Gabrielle Zacks, Dublin, CA (US); Dan Attema, Dublin, CA (US); Chris Burns, Dublin, CA (US); Tim Surber, Dublin, CA (US); Fred Ouding, San Leandro, CA (US); Jochen Straub, Dublin, CA (US)

(73) Assignee: Carl Zeiss Meditec, Inc., Dublin, CA (US)

(21) Appl. No.: 19/341,552

(22) Filed: Sep. 26, 2025

Related U.S. Application Data
(60) Provisional application No. 63/700,187, filed on Sep. 27, 2024.

(51) Int. Cl. A61B 3/12; A61B 3/00; A61B 3/10; B25J 15/04

(52) U.S. Cl. CPC A61B 3/1225; A61B 3/0083; A61B 3/102; B25J 15/0408

(57) ABSTRACT

An ophthalmic system/device (e.g., OCT or fundus imager) provides easy alignment for a patient with no, or minimal, assistance from a technician. This is achieved in a low cost device by using various patient-customized mechanisms that provide an initial rough alignment that brings the patient within an expected alignment range of the device. The ophthalmic device then provides a fine alignment by means of a custom mechanical guide operated by a single motor.

### CROSS-REFERENCE TO RELATED APPLICATIONS

This application claims priority to, and the benefit of, U.S. Provisional Ser. No. 63/700,187, filed Sep. 27, 2024 and titled “OPHTHALMIC SYSTEM,” which is incorporated by reference herein in its entirety for all purposes.

### FIELD

The present disclosure is generally directed to various mechanical issues related to an ophthalmic system, such as an optical coherence tomography (OCT) system and/or fundus imaging system (fundus imager). In particular, the present disclosure is directed to a low-cost ophthalmic system (e.g., OCT).

### BACKGROUND

There is a desire for a low-cost way to move a set of optics (of an ophthalmic device such as an OCT or fundus camera) from one eye to another while clearing the nose of a patient. The method should work with a range of interpupillary distances (IPDs), be accurate, and be repeatable. The method should be automated, and eliminate or minimize the use of an operator/technician. Devices typically accomplish this with an XYZ stage, which is expensive, or with the aid of an operator/technician manually aligning the optics to each eye (not an option when an objective is to have no operator).

There is also a desire for a low cost, smooth, reliable way to scan a sample arm (e.g., of an interferometric system, such as an optical coherence tomography, OCT) within +/−3 mm from its central position. Previous methods used linear motors, which are costly. Additionally, a motor would have to change directions twice per cycle, which takes time, and may not be smooth.

In regard to ocular lens adjustment, there is a desire for a set of two ocular lenses to be translated in the Z direction of XYZ space (e.g., towards and away from the patient) within a total range of +/−5 mm in roughly 0.25 mm increments (½ diopters). This sets the focus of the device and is adjusted according to the patient's refractive error. The goals are high accuracy and repeatability, ease of use in adjustment by a low-skilled technician, and low cost. In a typical ophthalmic device, this is done with a motorized stage. However, in a low-cost device, it is desirable to accomplish the same effect without a motor or linear guide.

Additionally, an ophthalmic system/device may the mounting of a double prism securely, accurately, and in a compact space. This may pose some issues addressed herein.

The ophthalmic system may be embodied in an OCT system, which may include a pupil camera and/or fundus camera. The present OCT system provides custom face support (e.g., a face mask customized to a patient's face), and this custom face support may be produced with additive manufacturing. Other manufacturing processes are currently found to be cost prohibitive. However, additive manufacturing is limited as to the accuracy it can achieve.

### SUMMARY

The above goals are met in a method/system for operating or constructing an ophthalmic system/device. Our system uses a patient-customized “smile” (part with an arc shaped path) to guide the optics head into roughly the right place. Then there is a patient-customized “slug” (part with a kinematic cone) to finely guide the optics head (e.g., of an OCT) to the right location. This system uses polar coordinates instead of cartesian coordinates to define motion. This system accomplishes this motion with 1 DC motor. This approach results in a system with repeatable and accurate location of the optics head, low cost, and automation.

Some characteristics of a coarse alignment system may include: Single motor driving all motion to custom X, Y, Z positions; Pogo motion/polar coordinates; Smile interpupillary distance (IPD) customization; Creates path guidance for any set of pupil coordinates; Allows loose tolerances in motion mechanism; Dorsal fin to constrain motion about Y′ (vertical) axis; Worm gear for binocular motion; Prevents the use of a separate brake; Mounting with belt drive to prevent loading the motor axially; Overdrive for binocular motion to ensure full seating of the kinematic ball into the kinematic cone; Current sensing for overdrive motion and safety; and Smooth and reliable transitions from coarse motion (smile) to fine alignment (kinematics) and back.

Some characteristics of fine an alignment system may include: Kinematic ball in the optics head interfaces directly with the kinematic cone in the mask to reduce tolerance stack; Kinematic ball adjusts to optics; and Kinematic cone location customized for precise location of pupil coordinates.

Some characteristics of shutters in some embodiments may include: Cam-actuated shutters and Seals on shutters to keep out dust and debris.

In embodiments, a cam is attached to the shaft of a motor turning at a constant angular velocity. The cam has a radius that increases linearly as the angle increases. Therefore, the motor's constant angular velocity is translated into constant linear velocity in the cam follower. To have equal velocities in both directions, the cam's radius would increase and decrease at the same rate. However, if it's a greater priority to have fast scanning and only one direction of motion is important, then the cam's radius could increase and decrease at different rates for a fast and slow direction (e.g., scan direction), all with constant angular velocity of the motor. In between the zones of increasing and decreasing radius is a smoothed area with no flat spots to ensure that the cam follower can remain smoothly in contact with the cam without jumping or sudden accelerations. In embodiments, a cam is used to turn constant angular velocity into constant linear velocity in two directions. The outbound and inbound directions can have equal or different linear velocities. To applicant's understanding, there aren't other cams being used in this context (scanning across the eye) at either a constant speed or one speed on the way out and another on the way back.

### Ocular Lens Adjustment

There are several aspects to the ocular lens adjustment mechanism, including: Linear motion is guided with a 3-line contact dovetail interface; this dovetail interface is maintained by a sprung gib; linear motion is actuated manually with an adjustment screw that is turned with a custom tool; location of the ocular lenses is measured with a magnet detected by a Hall effect sensor; and there is an optional brake that is actuated by the adjustment screw tool to prevent the adjustment screw from backdriving.

Some advantages of the some embodiments include accurate and repeatable ocular lens placement with simple parts and no motor. Additionally, adjustment is possible by a low-skilled technician, yet it cannot be tampered with by the patient. The ocular lens placement does not drift or change with time—it is stable and does not rely on electronics to hold its position. This is optimal in a patient-customized device, where the refractive error is set once and may stay constant for several years.

Some novel features of the approach include: using a 3-line contact dovetail instead of a linear guide; Gib for keeping dovetail in place; Retractable spring-loaded gib; Pitch of adjustment screw matched to technician's hand resolution for application; Hall effect sensor for measuring ocular lens position; Automatic mechanical brake for holding ocular lens position; and Self-engaging ocular adjustment tool for keeping brake disengaged when adjusting.

As stated above, an embodiment of the present ophthalmic system may mount a double prism securely, accurately, and in a compact space. The present solution may include a system that consists of a tightly toleranced part with 3 point contacts, and a sheet metal clamp to hold the optic against 6 point contacts.

### Prism Mounting with 6 Points

This new mounting system defines the position and orientation of the prism with surfaces 1 and 2 as shown in the optical model, below. The system consists of a tightly toleranced part with 3 point contacts each on surface 1 and surface 2, and a sheet metal clamp to hold the optic against the 6 point contacts. Characteristics of the present approach are: Mounting with 6 points of contact but using tolerancing to keep the optic in the correct position; Mounting with only the front prism surfaces; and Compliant spring mount for holding optic against hard mounting points.

As mentioned above, the present disclosure makes use of a multi-step manufacturing process to manufacture customized patient face mask supports using a low cost, additive manufacturing, but still provide high precision and accuracy of kinematic cone placement. A summary of the multi-step manufacturing process is as follows.

### Process

1. Produce mask with additive manufacturing
2. Mount mask in jig block
3. Measure mask and known jig block fiducials
4. Locate mask within jig block from those measurements
5. Post-machine mask relative to known jig block location and mask location within that block
6. Use post-machined features to accurately place kinematic cones

High accuracy is desired in this application because the tolerance stack for aligning the beam to the eye is quite tight, and the tolerance of the face surface of the mask to the cone location directly contributes to that stack. Two possible ways to tackle this challenge include: (one) machining the entire mask in one process from a blank, and (two) the method described in more detail below. Machining the mask from a blank is cost prohibitive—first investigations showed that it would cost 5× the cost of the entire printed mask to make the blank alone. The cost for a blank is high due to people's face geometries being very different, to the point where there would either have to be thousands of different blanks for different combinations of feature sizes or a very thick blank (difficult to manufacture and including a large amount of material). To overcome these limitations, the preferred approach is to use in-process measurement of the mask. The presently preferred process enables accurate post-machining without a known physical datum in the part. This is crucial with additive manufacturing parts because there are no “perfect” datums from which to make measurements. In the present mask, the datum relative to what may be post-machine is the entire patient's face surface. The present process can be automated to the point where, in operation (i.e., in the field), a technician/operator may mount the mask in a jig and press a “start” button.

In regard to the use of a face mask customized to a patient's face or other custom face support produced with additive manufacturing, it has been found that additive manufacturing is limited as to the accuracy it can achieve. For example, a preferred face mask may include two kinematic cones that are placed in the forehead area at very high accuracy—higher than can be achieved with additive manufacturing. These cones are what locate the optics to the pupils, so if they are not placed accurately, the optics will be misaligned to the patient. To overcome these limitations, the present disclosure makes use of a multi-step manufacturing process.

Other features, functions and attainments together with a fuller understanding of the disclosure will become apparent and appreciated by referring to the following description and claims taken in conjunction with the accompanying drawings.

Several publications may be cited or referred to herein to facilitate the understanding of the present disclosure. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings wherein like reference symbols/characters refer to like parts:

FIG. 1 shows the main components of the internal moving parts of the coarse alignment system.

FIG. 2 shows the spherical bearing placement in the structural housing.

FIGS. 3A and 3B show the approximate range of motion of the assembly, e.g., cones of motion or conical motion (herein called pogo motion), as viewed from the front (FIG. 3A, left) and the side (FIG. 3B, right).

FIG. 4 shows the smile assembly consisting of a custom smile track pressed into a generic structural frame.

FIG. 5 shows the smile assembly 19 at the top of the device, highlighted in blue.

FIG. 6 shows the main gearbox components of the gear box system that interface between the motor and smile.

FIG. 7 shows the gearbox of FIG. 6, as placed in the system with the smile, structural features, and joystick.

FIG. 8 provides a more detailed view of worm wheel 27 and pin drive plate 29, including two torsion springs.

FIG. 9 shows further detail of the drivetrain, including the coupling of the motor to the timing belt, to the worm, to the worm wheel.

FIG. 10 shows the main components of the fine alignment system.

FIG. 11 shows kinematic ball 31 (pink) protruding from the Internal Moving Parts, with the beam's approximate location 39 shown in blue.

FIG. 12 provides a close-up view of the kinematic ball adjustment mechanism.

FIG. 13 shows the precision-machined cone.

FIG. 14 shows the interface between the kinematic ball and the cone.

FIG. 15 shows mask 35 with two kinematic cones 33A and 33B (pink) installed.

FIG. 16 provides a view of the front face plate, as viewed from inside the ophthalmic device, with the oculus dexter (OD or right eye) shutter in the opened position and the oculus sinister (OS or left eye) shutter in the closed position.

FIG. 17 shows the shutter assembly via a translucent view of the front face plate (as viewed from outside the device) in the context of the device structure.

FIG. 18 show a shutter 43 with rubberized edges 45 highlighted in blue.

FIGS. 19A, 19B, 19C, and 19D show steps in the actuation of the shutters under the face plate, as viewed from outside the ophthalmic device (with the face plate being shown translucent for illustration purposes, but with the understanding that the face plate is opaque).

FIG. 20 is an example of how to define an even return cam.

FIG. 21 illustrates the shape of cam following the values of FIG. 20, where the rate of radius decrease from 0° to 180° is equal to the rate of radius increase from 180° to 360°.

FIG. 22 shows the cam of FIG. 21 modified to avoid discontinuities in its shape.

FIG. 23 shows the resultant cam after modification.

FIG. 24 is an example of how to define a fast return cam with the inbound velocity 2× the outbound velocity.

FIG. 25 shows the resulting cam shape following the values of FIG. 24.

FIG. 26 show the cam shape after applying rounds to the cam of FIG. 25.

FIG. 27 provides some metrics to the cam of FIG. 26.

FIG. 28 shows an example of a 3-line contact dovetail interface for the present ocular lens adjustment system.

FIG. 29 illustrates a mechanism for achieving three lines of contact, including a gib for maintaining the dovetail slider in place.

FIG. 30 augments the mechanism of FIG. 28 with the addition of a gib and position adjustment mechanism.

FIGS. 31 and 32 show an adjustment screw and custom tool for providing fine pitch movement for the dovetail.

FIG. 33 shows a hall effect sensor, in accordance with the various embodiments.

FIGS. 34-38, 39A and 39B illustrate the addition of an optional motion break, e.g., a screw rotation lock, to prevent undesired motion of the ocular lenses.

FIGS. 38, 39A and 39B show the ocular adjustment tool suitable for use with the optional motion break of FIGS. 34-37.

FIG. 40 shows a prism model.

FIG. 41 shows a prism mount from Thorlabs™.

FIG. 42 shows a prism mounting mechanism in accord with an exemplary embodiment of present invention.

FIG. 43 provides a side view and a bottom view of a preferred mount using six point contacts, three on each of the surfaces of interest.

FIGS. 44 and 45 provide additional projection and perspective views of the prism mount.

FIG. 46 shows exemplary goniometers that may be oriented toward a patient.

FIG. 47 provides an exemplary application of fixation.

FIG. 48 shows a patient's head positioned within an ophthalmic device's patent support, including outer Canthus marks.

FIG. 49 illustrates rotating an ophthalmic device (e.g., iris cameras/fixation/3D scanner assembly) until an unobstructed view of the pupil is obtained.

FIG. 50 illustrates a two-piece jig block, in accordance with the various embodiments.

FIG. 51 shows the use of a Coordinate Measuring Machine (CCM) with the jig of FIG. 50.

FIG. 52 shows using the nose (particularly the nose bridge) as a reference for determining the eye opening width.

FIG. 53 shows a method for making the nose piece as wide as possible without going into the inner corner of the patient's eye.

FIG. 54 show an example of a resultant nose pieces on a face mask (e.g., front view and back view).

FIG. 55 illustrates the adjusting of face length.

FIG. 56 shows the adjusting of size between long and short faces.

FIGS. 57 and 58 show the result adjusting for face length.

FIG. 59 shows a method of determining kinematic cone locations for a mask.

FIG. 60 shows the adjusting of an ophthalmic device for different refractive errors, in accordance with the various embodiments.

FIG. 61 shows how the thickness of the patient-customized face mask can be adjusted to compensate for positioning of the ocular lens due to a patient's refractive error to maintain a constant working distance (e.g., 17.1 mm) between the ocular lens apex and patient's corneal apex.

FIG. 62 highlights the three main parts/portion of a mask template, in accordance with the various embodiments.

FIGS. 63-67 show various stages in the construction of custom patient face mask, including some front and back view pairs.

FIG. 68 is an example of the handlebar feature, shown in the template from the top, and consisting of a horizontal bar protruding from each side of the forehead portion of the mask.

FIG. 69 shows the placement of the mask onto the ophthalmic device.

FIG. 70 shows a blue highlighted surface that fits inside the notch, constraining the mask from moving.

FIGS. 71 and 72 show the final steps in coupling the face mask to the ophthalmic device.

FIG. 73 illustrates part of the process of mounting the face mask to an ophthalmic device, wherein this figure shows the process beginning with removing its cover.

FIG. 74 illustrates part of the process of mounting the face mask to an ophthalmic device, wherein this figure shows the mask being hooked on at the bottom by putting the upside-down U-shaped opening onto the chin bar.

FIG. 75 illustrates part of the process of mounting the face mask to an ophthalmic device, wherein this figure shows the mask being pivoted into the device from the bottom until the handlebar is in place.

FIG. 76 illustrates part of the process of mounting the face mask to an ophthalmic device, wherein this figure shows that, when the handlebar is locked in place, the mask lifts off slightly from the chin pin in the vertical direction, but still provides support for the mask from rotating counterclockwise, when the patient leans into it.

FIG. 77 shows this partial compression of the face to compensate for a patient's face being compliant (tissue is malleable).

FIG. 78 is an example of a patient with an off-center nose resulting in an uneven nosepiece on the mask, as seen here from the top.

### DESCRIPTION

### Binocular Device

The binocular motion of the present ophthalmic system is guided by a coarse alignment system and a fine alignment system. The goal of the coarse alignment system is to deliver the optics head within the acceptance extents of the fine alignment system. The coarse alignment system is designed to be made from low-precision components, such as injection molded plastic parts. This reduces cost of the system considerably.

### Coarse Alignment System

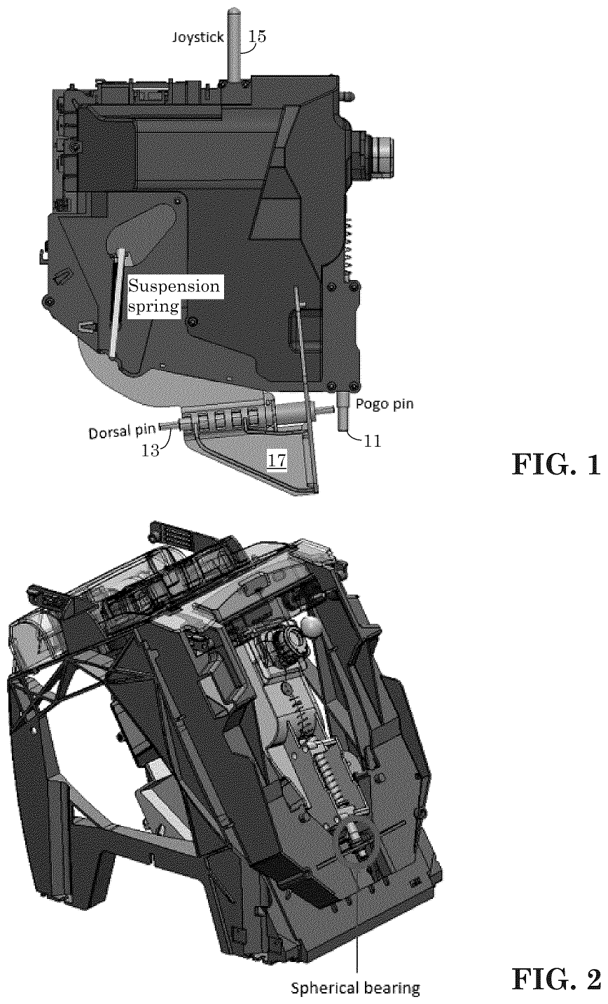

The coarse alignment system consists of the internal moving parts, smile, and gearbox. FIG. 1 shows the main components of the internal moving parts of the coarse alignment system. In this example, the optics head (e.g., of an optical coherence tomography system, OCT) and electronics are contained in the internal moving parts. The moving parts are supported in the device structure by a pogo pin 11 and a dorsal pin 13, and are steered by a joystick 15.

The dorsal pin goes through the dorsal fin 17 (green), which constrains motion of the internal moving parts so they can't rotate about the Y′ axis (Y′ is vertical as shown above, but the angle will change depending on the position/angle of the optics head, so we'll call it Y′). The dorsal pin 13 is supported by stationary structural parts on either end. The dorsal fin 17 can pivot on this pin 13 and is trapped inside the internal moving parts. Within the internal moving parts, the dorsal fin 17 can slide in the Y′Z plane so that motion is free within that plane. There is a pair of extension springs between the internal moving parts and the dorsal fin 17 to act as a suspension system and take up shock loads between the external world and the internal moving parts. This protects the optics and electronics against bumps and light drops in shipping and normal use in a home setting.

The pogo pin 11 can slide vertically within the internal moving parts to allow motion in the Y′ direction. There is a compression spring to bias the internal moving parts upwards, providing further suspension to the system. The bottom of the pogo pin sits in a spherical bearing to constrain motion of the pin 11 at that point in X and Z. FIG. 2 shows the spherical bearing placement in the structural housing. The spherical bearing's housing is statically mounted in the non-moving structural parts of the present ophthalmic system.

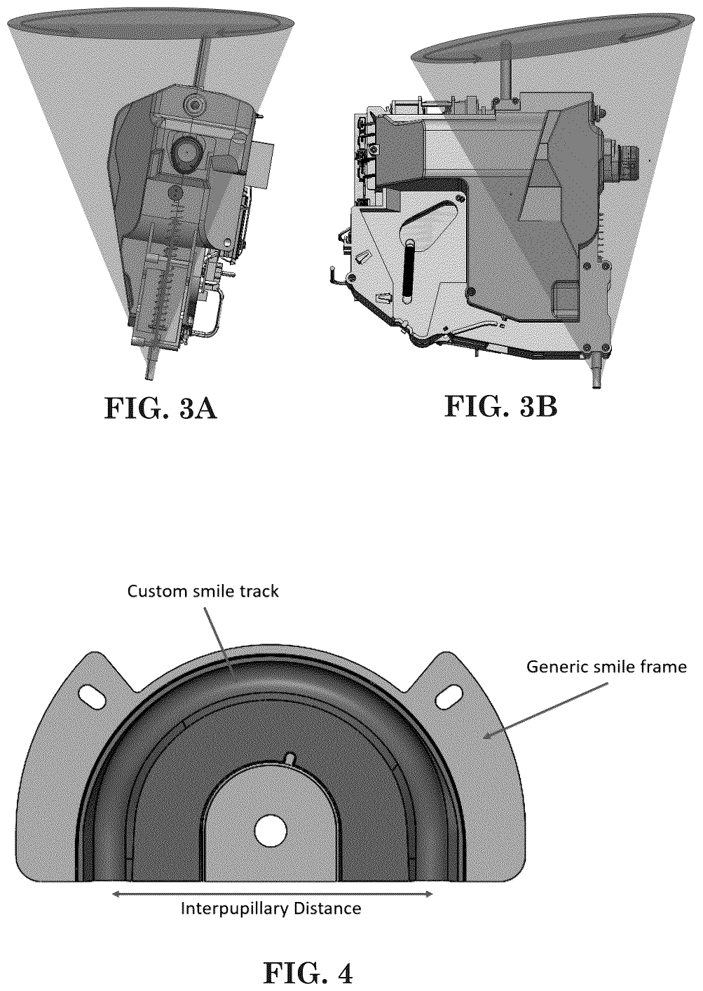

The combination of the dorsal fin 17 and dorsal pin 13 and the pogo pin 11 and spherical bearing mean that the optical axis is always pointing in Z dimension (towards the patient's eye) and the entire internal moving parts assembly can move in a cone shape+translate up/down in Y′. By use of the joystick, one can move the assembly throughout its range of motion. FIGS. 3A and 3B show the approximate range of motion of the assembly, e.g., cones of motion or conical motion (herein called pogo motion), as viewed from the front (FIG. 3A, left) and the side (FIG. 3B, right). This pogo motion is described in polar coordinates.

SMILE—patient-customized part with an arc shaped path herein termed “smile” guides the optics head into roughly the right place.

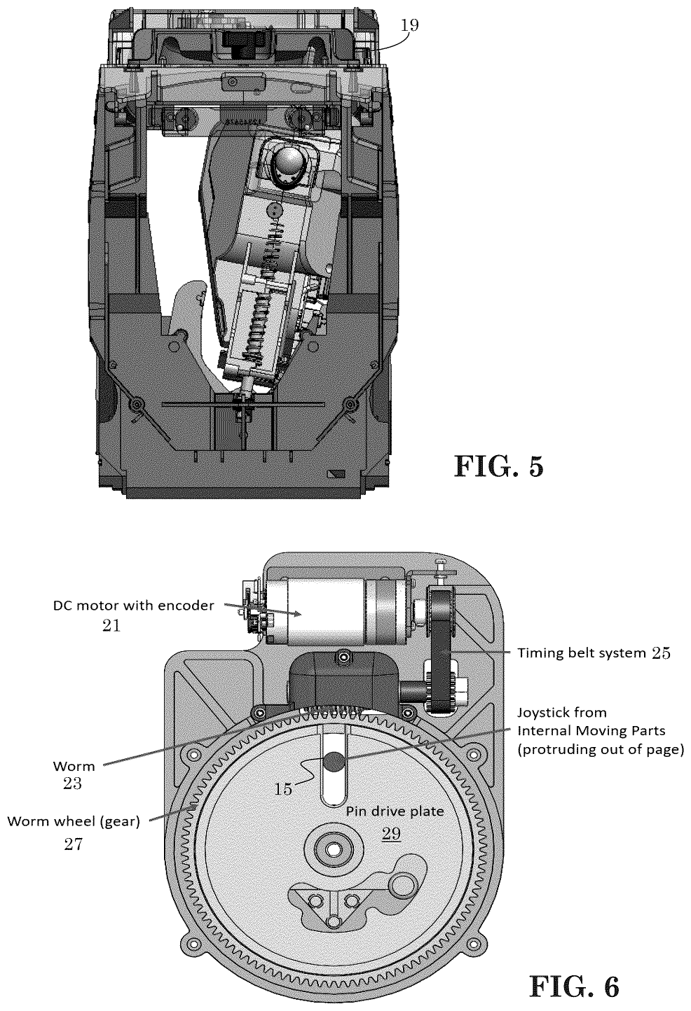

FIG. 4 shows the smile assembly consisting of a custom smile track pressed into a generic structural frame. FIG. 5 shows smile 19 at the top of the device, highlighted in blue.

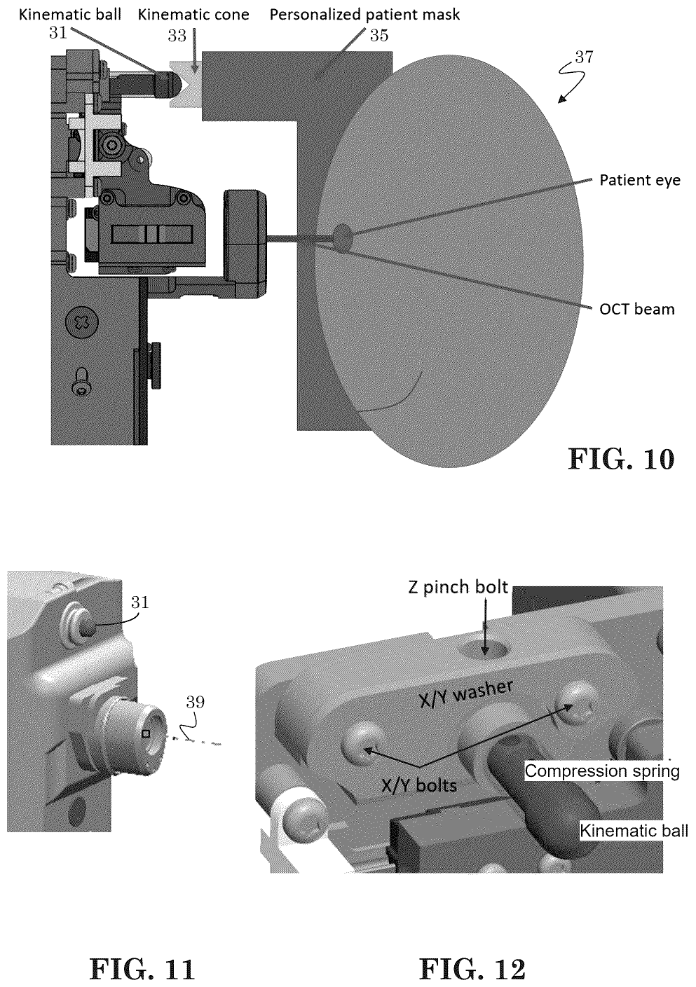

A goal of the present system is to move the optics head in a U shape between the eyes and around the nose. The motion is guided with a custom smile part that makes a track for the joystick the width of the patient's interpupillary distance (IPD). This smile deposits the optics head within a range of the kinematic cones to accept the kinematic ball (see FIG. 10).

The reason that the smile is custom is that everyone has a different IPD, so this part is made to match that patient's IPD. However, because the smile is part of the coarse alignment system, the smile does not need to be made very accurately. The present exemplary application uses additive manufacturing (3D printing) because it is easily customized.

### Gearbox

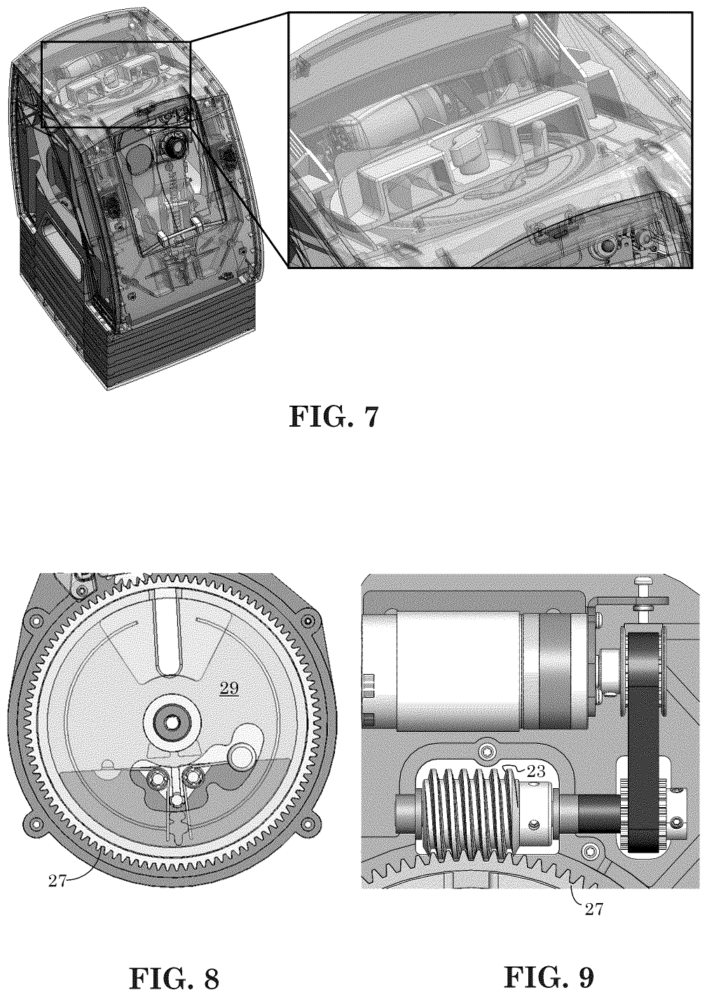

The smile 19 is what guides the motion path, but the system may include an interface between the motor and smile 19 to move the system along the smile guide (or smile track, see FIG. 4). That is done with a gearbox system. FIG. 6 shows the main gearbox components of the gear box system that interfaces between the motor and smile 19. Highlighted are the DC motor 21 with encoder, which engages with a worm (gear) 23 via a timing belt system 25. Worm 23 is coupled to worm wheel (gear) 27. Also shown is a pin drive plate 29 and joystick 15. FIG. 7 shows the gearbox of FIG. 6, as placed in the system with the smile, structural features, and joystick. FIG. 8 provides a more detailed view of worm wheel 27 and pin drive plate 29, including two torsion springs. FIG. 9 shows further detail of the drivetrain, including the coupling of the motor to the timing belt, to the worm, to the worm wheel.

The design goals were reducing cost by accomplishing all motion with a single motor, and flexibility to adapt to different IPDs with the same hardware. The joystick 15, which steers the Internal Moving Parts about the bottom spherical bearing, interfaces with the gearbox system.

The joystick 15 protrudes upward through a slot in the pin drive plate 29. The rotation of the pin drive plate 29 then moves the joystick 15, steering the entire system. The slot in the pin drive plate 29 allows the joystick 15 to translate radially (see FIG. 6) and to stay within the track created by the smile (see FIG. 4), allowing for different IPDs.

The pin drive plate 29 sits inside the worm wheel 27 and is driven by the worm wheel 27 via a bump that contacts the two torsion springs.

Normally there is not very much resistance in the system, so the bump is slightly (but not fully) compressing one of the torsion spring legs as the optics head travels in that direction. When the kinematic ball makes contact with the kinematic cone (see FIG. 10), the Internal Moving Parts cannot travel any further. Right after this happens, the motor is still running. This means that the worm wheel 27 keeps turning and the torsion spring keeps compressing, driving up the current in the motor. The motor controller is programmed to stop at a certain current, at which it is pretty certain that the kinematic ball is fully seated. This ensures that one does not hit a hard stop and bounce out—the overdrive springs make sure that the kinematic ball is fully seated in the kinematic cone before the motor stops.

The current monitoring is also a safety feature. The encoder on the motor tells us where to expect the joystick to be at any given part of the path. If the current starts rising above a certain threshold before it's near the ends of the smile, that tells the system that something is wrong and to stop the motion.

The gear train is: motor>motor gearbox>pulley>belt>pulley>worm>worm wheel. This provides a large gear ratio which means that with a relatively weak DC motor, the binocular system can still be moved with a lot of torque, though at a low speed. The second purpose of having a worm in the system is that a worm cannot be back-driven (i.e. Driven by the worm wheel), which acts as a brake in the system while it is not in use. This protects the components in shipping or other transportation of the device.

Driving a worm induces axial loads on its shaft. If the worm were directly coupled to the motor shaft, these axial loads could cause premature motor wear/failure. To prevent this, the worm is mounted via flanged bushings directly into the assembly's plastic housing and coupled to the motor with a timing belt. The timing belt is tensioned with a screw on the motor mount that is in-line with the belt to prevent twisting. The motor mount's attachment to the plastic housing is on slots to guide this tensioning, and then is tightened into place.

### Fine Alignment System

FIG. 10 shows the main components of the fine alignment system. The fine alignment system is designed to accept the optics head from the coarse alignment system (discussed above), and then accurately and repeatably align the optics to the patient's eyes. The goal of the fine alignment system is to reduce the number of high precision/accuracy parts to a few key components to reduce tolerance stack up and cost.

The fine alignment system consists of the kinematic ball 31, the kinematic cone 33, the (e.g., personalized) mask/slug 35, and the patient 37.

Due to tolerances within the optics head, the beam is not necessarily exiting the ocular lens in the center. There is a tolerance zone as to where the beam ends up. There is tooling used to calibrate the kinematic ball 31 to the beam so that the ball center is a standard distance from the beam. FIG. 11 shows kinematic ball 31 (pink) protruding from the Internal Moving Parts, with the beam's approximate location 39 shown in blue.

FIG. 12 provides a close-up view of the kinematic ball adjustment mechanism. The kinematic ball 31 consists of a precision-machined sphere at the end of a smooth shaft. The kinematic ball 31 is sprung with a compression spring in Z around the shaft. It has a pinch bolt in Z and can float in X and Y under a washer that is clamped tight by two screws. The tooling has a conical receiver (i.e., kinematic cone) for the kinematic ball.

The way it is aligned is by use of three screw. X, Y and Z screws (or bolts) are loosened, driving the kinematic ball into the tool receiver. The beam center is found with a sensor. This sensor is calibrated to a known distance from the receiver. Then the receiver moves the kinematic ball 31 until it is aligned with the beam. Then the screws are tightened, so the kinematic ball location will not move. Now the kinematic ball is aligned to the beam.

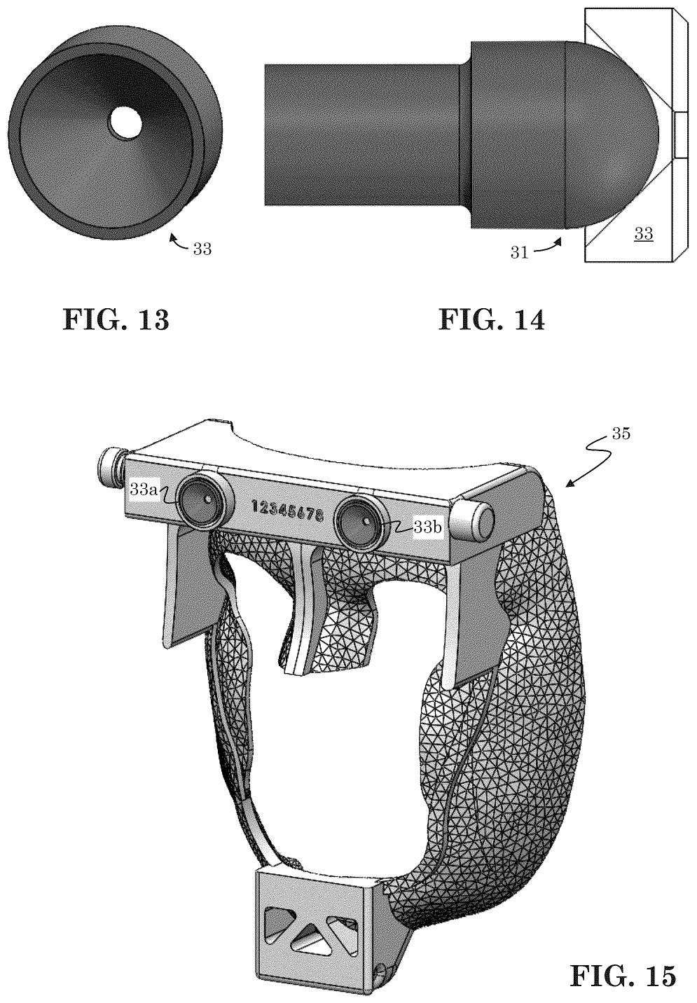

The next step in aligning the beam to the patient's eye is the interface between the kinematic ball 31 and kinematic cone 33. FIG. 13 provides a close-up view of the precision-machined kinematic cone 33. FIG. 14 shows the interface between the kinematic ball 31 and the cone 33. This kinematic cone 33 presses into the mask 35 (see FIG. 10), and is precision machined to have a smooth and accurate inside surface. This inside surface is cone shaped, which means that the spherical end of the kinematic ball 31 will end up in the same X, Y, Z location (the center of the cone) every time it is placed in it.

Two cones are pressed into the forehead of the mask 35 according to the patient's pupil locations. FIG. 15 shows mask 35 with two kinematic cones 33A and 33B (pink) installed. When the mask is being designed and manufactured, the hole locations for the cones are calculated according to the X, Y, Z coordinates of each pupil relative to mask (geometric and/or landmark) features.

The cone locations are calculated so that knowing the location of the pupils relative to the rest of the mask and the pogo geometry of the kinematic ball 31 and the beam 39, when the cone is placed in the mask, the patient's pupil will be aligned to the beam 39 for each eye.

The system described above allows for motion between two custom XYZ coordinates in an arc shape with only one motor and no linear guides. On a standard setup, one may include a three-axis motorized stage, which would increase the overall cost.

### Shutters

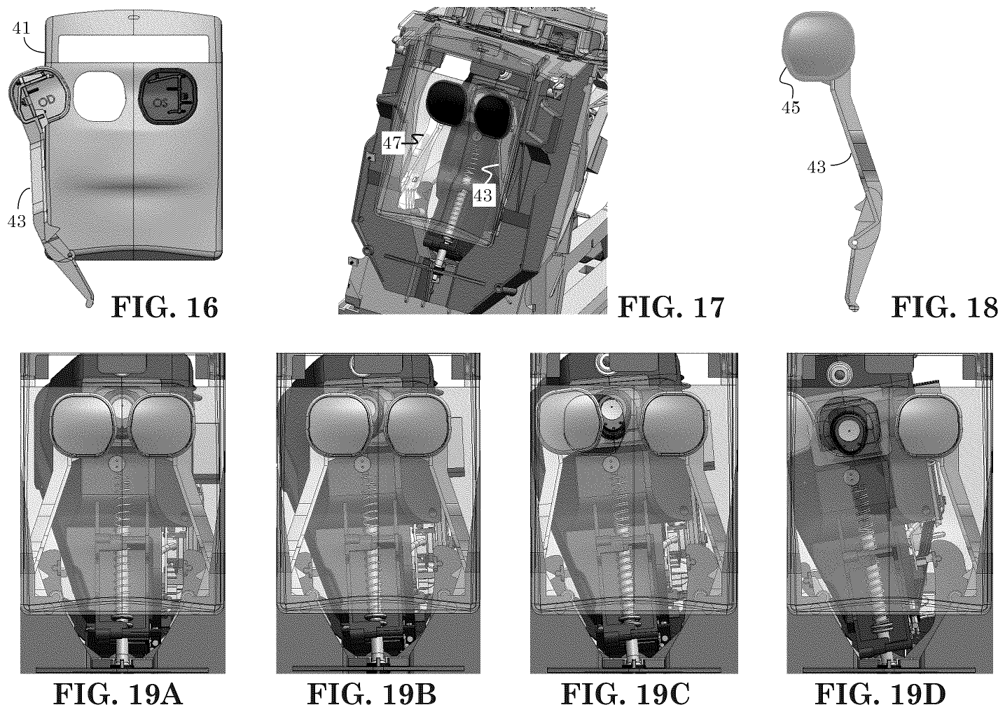

The front face plate of the ophthalmic device (OCT or fundas camera) has two round openings—one for each eye. FIG. 16 provides a view of the front face plate 41, as viewed from inside the ophthalmic device, with the oculus dexter (OD or right eye) shutter 43 in the opened position and the oculus sinister (OS or left eye) shutter (not shown) in the closed position, as indicated in pink. FIG. 17 shows the shutter assembly via a translucent view of the front face plate (as viewed from outside the device) in the context of the device structure. FIG. 18 show a shutter 43 with rubberized edges 45 highlighted in blue.

When a particular eye is being measured, the ocular lens pokes through the opening for that eye. However, when the eye is not being measured, that opening is closed (shuttered) to prevent dust/debris/fingers from getting in the device, and to block the other eye from seeing inside. This problem/objective is solved with a pair of shutters 43/47 that are actuated by cams on shutter arms and cam followers on a plate that travels with the Internal Moving Parts. When the Internal Moving Parts are approaching the OD or OS position, the cam follower makes contact with the cam and starts pushing the corresponding shutter arm out of the way. By the time the optics are in their final position, the corresponding shutter has been fully cleared out of the way. This is necessary because not only would the shutter block the optics from the eye, but the ocular lens also protrudes through the front face plate 41 to be at the proper working distance to the eye. Each shutter arm 43/47 is equipped with a return spring so that they are normally closed, and only opened when actuated by the Internal Moving Parts.

In order to keep dust and debris out of the inside of the device, the shutters have compliant rubberized edges 45 to make a seal with the front face plate 41.

FIGS. 19A, 19B, 19C, and 19C show steps in the actuation of the shutters under the face plate, as viewed from outside the ophthalmic device (with the face plate being shown translucent for illustration purposes, but with the understanding that the face plate is opaque). FIG. 19a shows a situation where Internal Moving Parts are centered (patient not under examination) and both shutters are closed. FIG. 19b shows that as Internal Moving Parts move towards left eye (OS), cam follower plate (green) makes contact with the cam on the shutter arm (white). In FIG. 19c, as the cam follower plate continues to move left, the shutter arm moves left as well, and in FIG. 19d the shutter fully clears the opening in the front face plate (translucent yellow) as the ocular lens pokes through the opening.

### Cam for Scanning Motion

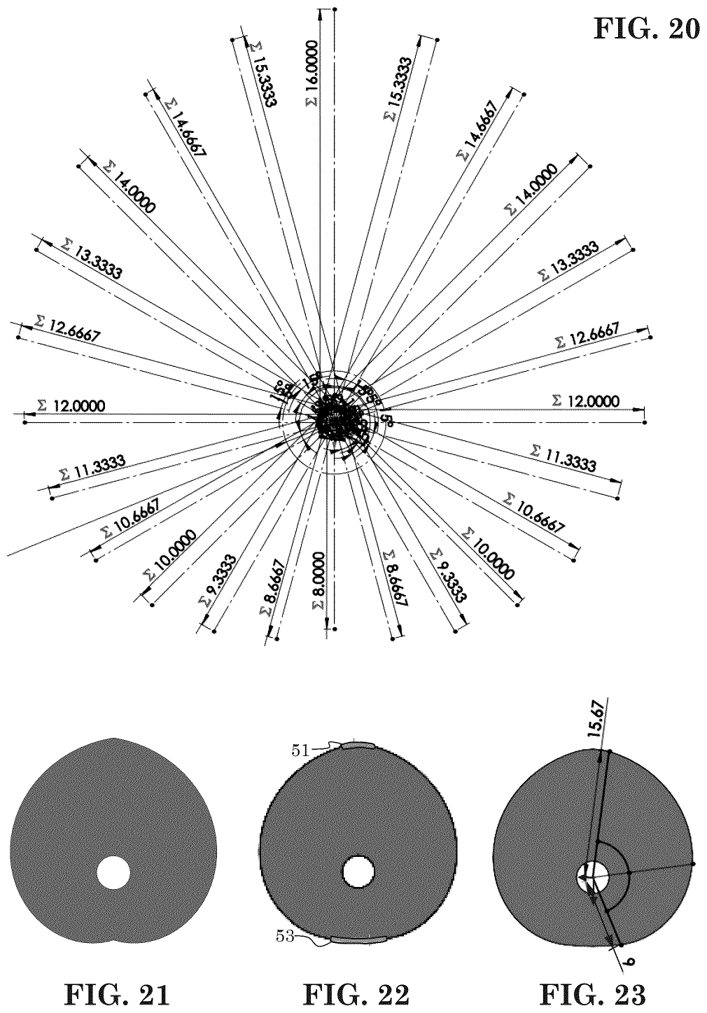

FIG. 20 is an example of how to define an even return cam. If one reads the values of FIG. 20 clockwise from the top, then from 0° to 180°, the radius of the cam is decreasing from 16 mm to 8 mm at a rate of ⅔ mm per 15°, or 4/90 mm per degree. The opposite is the case from 180° to 360°—the radius of the cam is increasing from 8 mm to 16 mm at a rate of ⅔ mm per hour 15°, or 4/90 mm per degree.

The rate of radius decrease from 0° to 180° is equal to the rate of radius increase from 180° to 360°, meaning the cam follower moves at the same linear speed on the way out and the way back given a constant motor angular velocity. FIG. illustrates the shape of cam following the values of FIG. 20, where the rate of radius decrease from 0° to 180° is equal to the rate of radius increase from 180° to 360°.

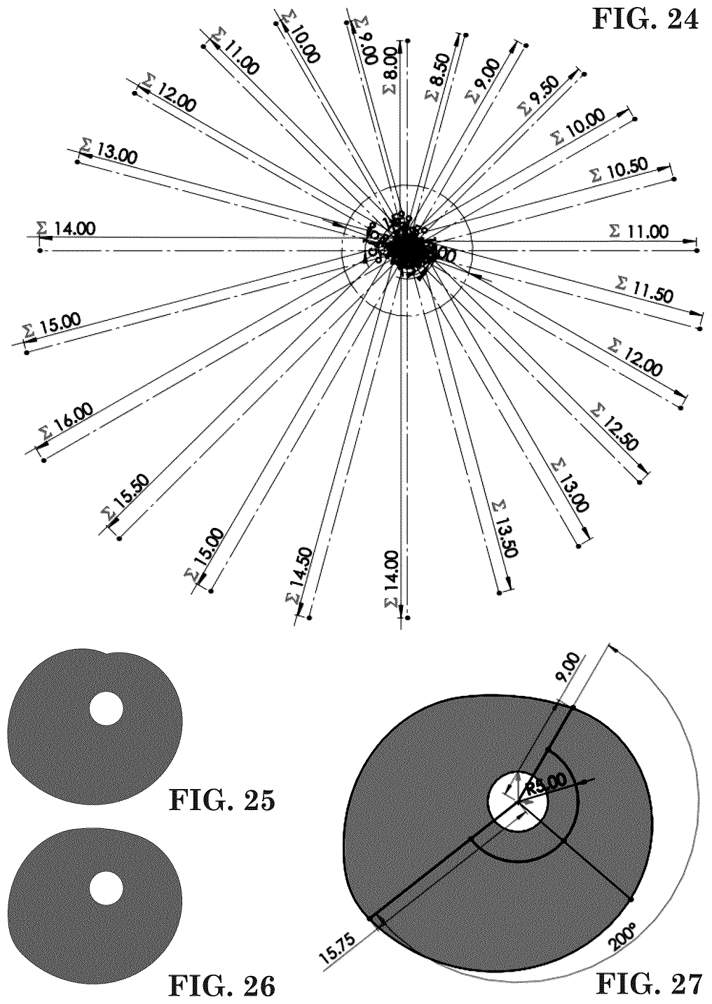

Practically, this would not be an ideal cam shape because there are discontinuities at the top and bottom, which a cam follower could not stay nicely attached to. FIG. 22 shows the cam of FIG. 21 modified to avoid discontinuities in its shape. FIG. 23 shows the resultant cam after modification. To keep the cam follower attached smoothly, the top 51 and bottom 53 are rounded so that there are no sharp changes in curvature (top) and the shape always remains convex (bottom). These rounds are shown below highlighted in blue.

These rounds should be considered when defining the starting and ending radii to ensure that the full linear range is achieved. In this example, there may be at least 6 mm of linear travel. Before rounds were added, there were 8 mm of linear travel in this cam. After rounds, that decreased to 6.67 mm.

FIG. 24 is an example of how to define a fast return cam with the inbound velocity 2× the outbound velocity. A similar concept of the radius increasing with the angle linearly holds true with this fast return. Again, reading the values clockwise starting at the top, the radius of the cam is increasing from 8 mm to 16 mm at a rate of 0.5 mm per 15°, or 1/30 mm per degree. This increase happens from 0° to 240°, or ⅔ of the circle. The cam then decreases in radius from 16 mm to 8 mm at a rate of 1 mm per 15°, or 1/15 mm per degree, twice the rate it was increasing. This decrease happens from 240° to 360°, or ⅓ of the circle.

The rate of radius decrease from 240° to 360° is twice the rate of radius increase from 0° to 240°, meaning the cam follower moves at twice the linear speed on the way back as on the way out given a constant motor angular velocity.

FIG. 25 shows the resulting cam shape following the values of FIG. 24, though the same considerations should be made for rounding the transitions. That is, FIG. 25 shows the cam shape before rounds. FIG. 26 show the cam shape after applying rounds to the cam of FIG. 25. FIG. 27 provides some metrics to the cam of FIG. 26. With the rounds excluded from scanning, the usable scanning portion of this cam is a 200° span that increases linearly by 6.75 mm.

### Ocular Lens Adjustment

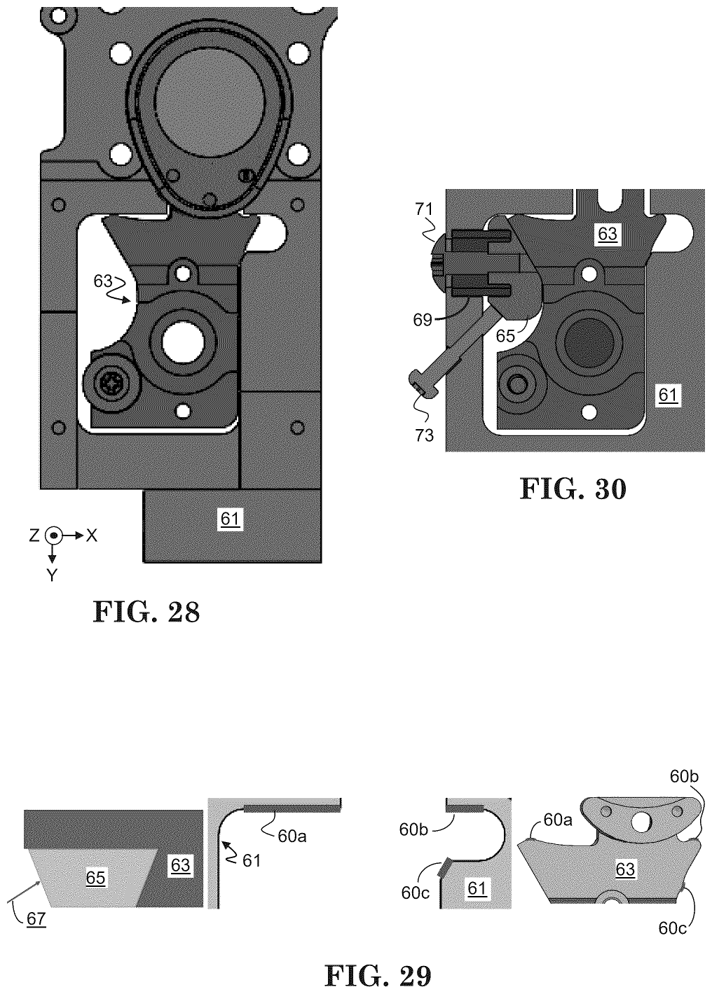

FIG. 28 shows an example of a 3-line contact dovetail interface for the present ocular lens adjustment system. The ocular lens adjustment system should move smoothly in the Z dimension (towards and away from patient, or into/out of the page in FIG. 28) while remaining stable in a constant X and Y location. This is typically done with a linear guide, but that would add cost and take up space that was not available in this setting.

Instead, the stationary housing 61 (grey) and moving slider 63 (teal) here have a dovetail interface so that a force from the opposite corner would drive the slider into the housing's corner in a very stable and repeatable way. To reduce friction and improve manufacturability, this is accomplished with distilling the dovetail down to three lines of contacts (e.g., “3 line contacts”), where only the 3 contact patches on each part may be tightly toleranced.

FIG. 29 illustrates a mechanism for achieving three lines of contact, including a gib for maintaining the dovetail slider in place. The 60a, 60b, and 60c are highlighted in red. FIG. 30 augments the mechanism of FIG. 28 with the addition of a gib and position adjustment mechanism.

In FIG. 29, a sprung gib 65 sits in an undercut section. For the dovetail slider 63 to stay in the proper X and Y position, it should include a force 67 coming from the opposite corner. This is achieved with the gib 65, or a wedge-shaped part that is forced into the dovetail slider 63 with a compression spring 69 (shown in blue in a cross-section in FIG. 30). Generally, a gib is a wedge-shaped component used to fill the gap between two moving parts. This compression spring 69 sits in an undercut in the housing 61 between the housing and the gib 65. The gib 65 is also threaded so that a wide-headed screw 71 can compress the spring 69 and retract the gib 65 while installing the dovetail slider 63. Otherwise, it would be difficult to slide in the dovetail slider 63 because the gib 65 would be in the way. There is also an optional set screw 73 that can be tightened when the ocular lenses are in the right position to add additional force for ensuring the dovetail slider 63 cannot be moved out of position if the ocular lens assembly is pushed on by a patient.

### Adjustment Screw and Custom (Ocular Adjustment) Tool

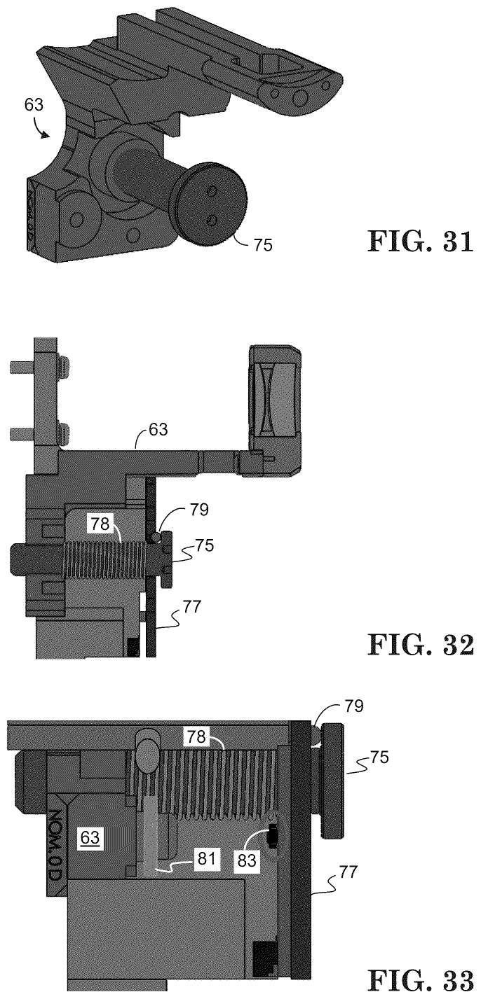

FIGS. 31 and 32 show an adjustment screw and custom ocular adjustment tool for providing fine pitch movement for the dovetail 63. The dovetail slider is moved in and out with a fine pitched screw 75 used as a lead screw. Generally, a lead screw converts rotary motion into precise linear motion, such as through the engagement of a rotating screw with a nut that slides along the screw. In the present case, the lead screw 75 threads directly into the center hole of the dovetail slider piece 63.

The pitch of the screw is matched to the application. Here, a ½ diopter resolution is desired, which comes out to about 0.25 mm of travel. The pitch of the screw used is 1 mm, because an operator turning the screw by hand can easily resolve a quarter turn increment.

M8×1 mm thread is a standard fine pitch thread, which makes it cheap to source for both the internal threads on the dovetail slider and the external threads on the adjustment screw. However, it is not a standard bolt-it has a low head (for nose clearance), and a 2-hole interface (see FIG. 31) for the custom (ocular adjustment) tool. These holes are very simple to manufacture—they can simply be drilled. But they provide the advantage that the custom ocular adjustment tool is not a standard-sized tool that a patient would have access to, making it tamper proof.

In various embodiments, the lead screw 75 is held stationary so that turning it only translates the dovetail slider 63. This is accomplished with a front plate 77 and compression spring 78 that pushes the dovetail back in Z (leftward in FIG. 32) until it contacts the purple front plate. There is an optional small metal ball 79 between the adjustment screw 75 head and plate 77 to make the contact a point rather than a surface. The purpose is to not be relying on a flat screw head/plate surface interface to keep the dovetail slider constrained by only the 3 dovetail line contacts. If it's just a point contact, then the screw head will adjust angularly to be in line with the dovetail contacts. This improves X and Y accuracy and consistency throughout the Z travel range.

### Hall Effect Sensor for Locating

FIG. 33 shows a hall effect sensor, in accordance with the various embodiments. The 0-diopter setting for each device will be in a slightly different position, due to optical tolerances throughout the system. Therefore, a way may exist to record a particular device's 0-diopter position (home position), and the other diopter positions relative to the home position. This is accomplished with a rare earth magnet 81 (highlighted blue) mounted to the moving dovetail slider 63 paired with a Hall effect sensor 83 (encircled in red).

The Hall effect sensor measures the strength of the magnetic field, which is related to the distance between the magnet 81 and the Hall effect sensor 83. This measurement is communicated to the main computer board, which converts the sensor reading to a diopter value. The device then communicates to the technician a diopter value, such as over audio and/or through a computer/cell phone app.

There may be a concern with placing a rare earth magnet 81 near the patient because strong magnets can have undesirable impacts on pacemakers. However, front plate 77, which is in between the Hall effect sensor and the patient, may be a magnetic stainless steel plate and deflect the magnetic field away from the patient.

### Optional Brake—For Use with Different Screw Pitches

It is not necessary with the screw diameter/pitch combination used in the present example (sufficient friction is present), but it is possible for another application that one would have a different screw that could be backdriven. In that case, one may use a brake to prevent undesired motion of the ocular lenses that could occur either spontaneously or if the ocular lens is pushed on.

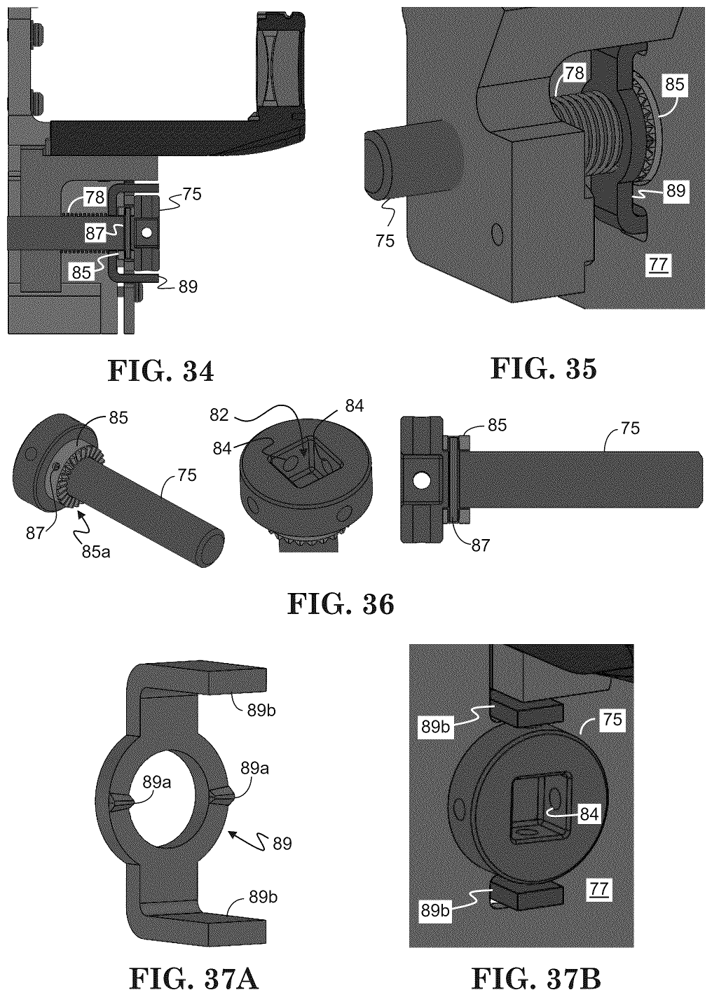

FIGS. 34-37 illustrate the addition of an optional motion break, e.g., a screw rotation lock. The brake described herein is always on, except for when the specialized tool, discussed above, is in the device. It's impossible to close up the device with the specialized tool in, therefore, the technician does not have to remember to set the lock. This happens automatically.

The adjustment screw mechanism consists of the adjustment screw 75 (green), a screw rotation lock 85 (yellow), and a roll pin 87 rigidly attaching the adjustment screw 75 to the screw rotation lock 85.

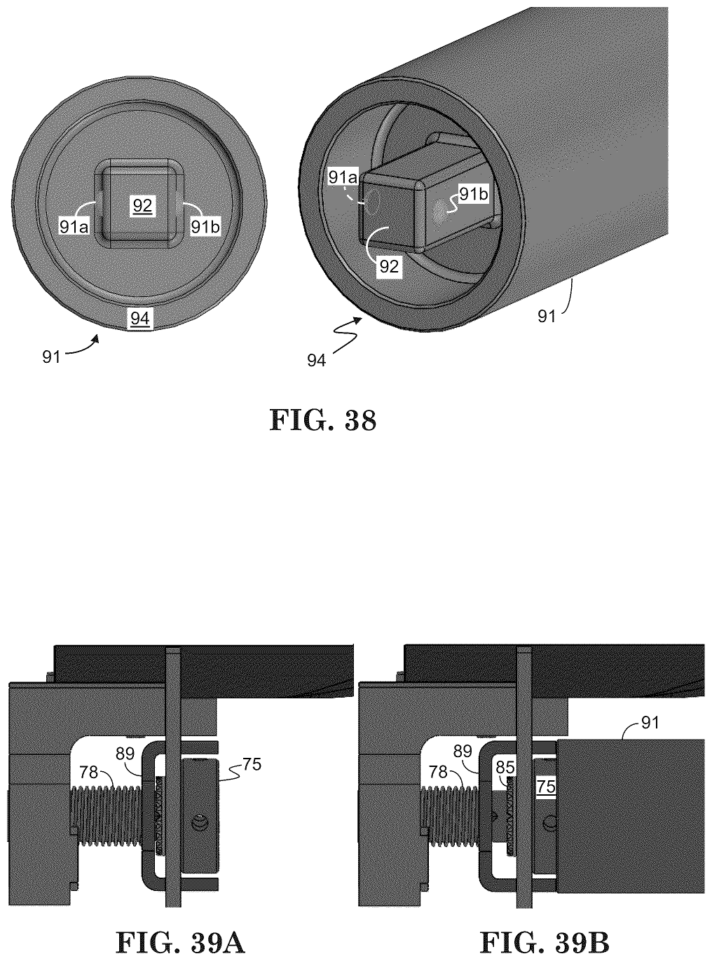

This adjustment screw 75 head has a square bit interface 82 with holes 84 on all sides to accept ball detents (91a/91b, see FIG. 38) on the ocular adjustment tool 91 shown in FIG. 38 (different from two drilled hole interface on the lock-free version, see FIG. 31). The screw rotation lock 85 has several small teeth 85a that engage with teeth 89a on the lock washer 89 to keep the screw's orientation locked when the lock washer 89 is engaged.

The lock washer 89 is a stamped piece of sheet metal that goes around the adjustment screw 75 between the compression spring 78 and the screw rotation lock 85. It has two tabs 89b that protrude towards the patient through holes in the front plate 77. The compression spring 78 ensures that the lock washer 89 stays pressed up against the screw rotation lock 85. When this happens, the teeth on the lock washer 89 and screw rotation lock 85 interlock, preventing rotation relative to each other. The orientation of the lock washer 89 is kept constant by the sides of the openings in the front plate 77, therefore the adjustment screw 75 also cannot rotate. The only way to disengage the lock washer 89 from the screw rotation lock 85 is to push the lock washer 89 back (away from the patient). While the lock washer 89 is pushed back, the adjustment screw 75 is free to be rotated.

FIGS. 38, 39A and 39B show the ocular adjustment tool suitable for use with the motion break of FIGS. 34-37. The lock washer 89 is pushed back with the ocular adjustment tool 91 (see FIG. 39B). In the version with the lock, this ocular adjustment tool 91 has a square 92 (see FIG. 38). Coming out of two sides of the square 92 are two ball detents 93a/93b (shown in blue below). Surrounding the square 92 is a pocket, and then an outer protruding ring 94. When the tool 91 is pushed all the way into the adjustment screw 75 head, the outer protruding ring 94 pushes the lock washer 89 back, disengaging it from the screw rotation lock 85 (see FIG. 39B). This involves compressing the compression spring 78 further, which would be tiring to hold for a long time while adjusting the lens position. That's where the ball detents 91a/91b come in—when the tool 91 is in far enough to disengage the lock washer 89, the ball detents 91a/91b engage with the holes 84 in the sides of the adjustment screw 75 head, locking the tool 91 to the adjustment screw 75 head. With the lock washer 89 disengaged and tool 91 engaged to the adjustment screw 75, the technician is then free to turn the tool 91 as usual to the correct diopter setting.

### Prism Mounting with 6 Points

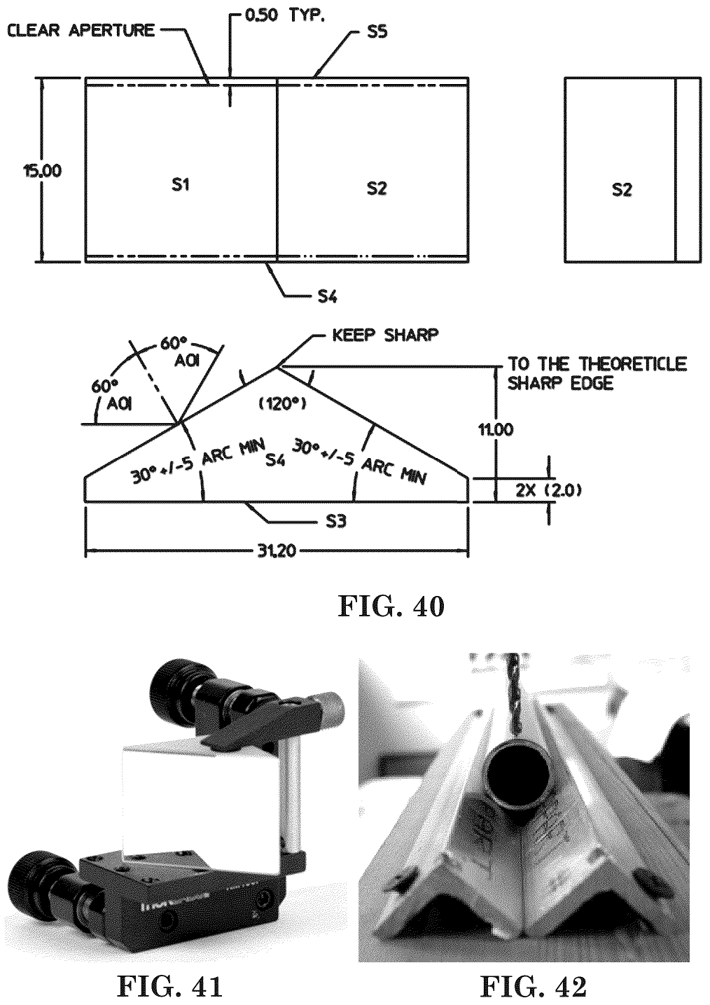

FIG. 40 shows a double prism model. The present ophthalmic system (OCT) mounts the double prism securely, accurately, and in a compact space. Existing solutions, such as prism mounts from Thorlabs™ or Edmund Optics™, are designed to hold and locate the prism from the top and bottom surfaces (S4 and S5) perpendicular to the prism faces (S1 and S2). This does not work for the present optic because S4 and S5 are not tightly controlled to S1 and S2. So, any mounting system for the present prism would have to rely only on S1, S2, and S3 for location and orientation.

Major optomechanics companies have several models of prism mounts as described above. For example, FIG. 41 shows is a prism mount from Thorlabs™.

The present new mounting system defines the position and orientation of the prism with surfaces S1 and S2 as shown in FIG. 40. The system may consist of a tightly toleranced part with 3 point contacts each on surface S1 and surface S2, and a sheet metal clamp to hold the optic against the 6 point contacts. Existing mounts are unable to accurately mount this optic without referencing the untoleranced surfaces, i.e., surface S4 and surface S5. The present novel approach allows the optic to be mounted with no, or minimal, added cost by having to tightly tolerance surfaces S4 and S5 relative to the optical surfaces, S1 and S2.

FIG. 42 shows a prism mounting mechanism in accord with an exemplary embodiment of present disclosure. This mounting mechanism is somewhat similar to a V block, in which a round part settles on two line contacts, as shown.

Rather than a round part, two flat surfaces are provided at a set angle to each other. A true kinematic mounting scheme would be three point contacts on one surface, and 1 point contact on the other surface. However, this results in an unbalanced set of forces, so the optic does not want to settle in place. This kind of truly kinematic mounting is not ideal here with such a large angle between surface S1 and surface S2, but it may work in another application where surface S1 and surface S2 are separated by a smaller angle.

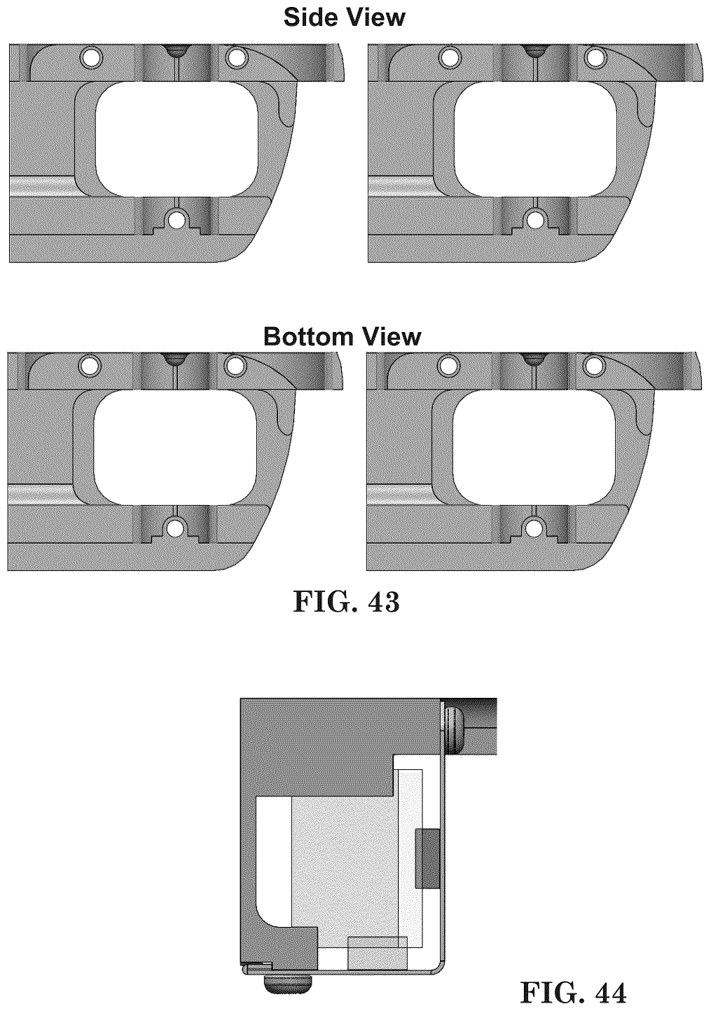

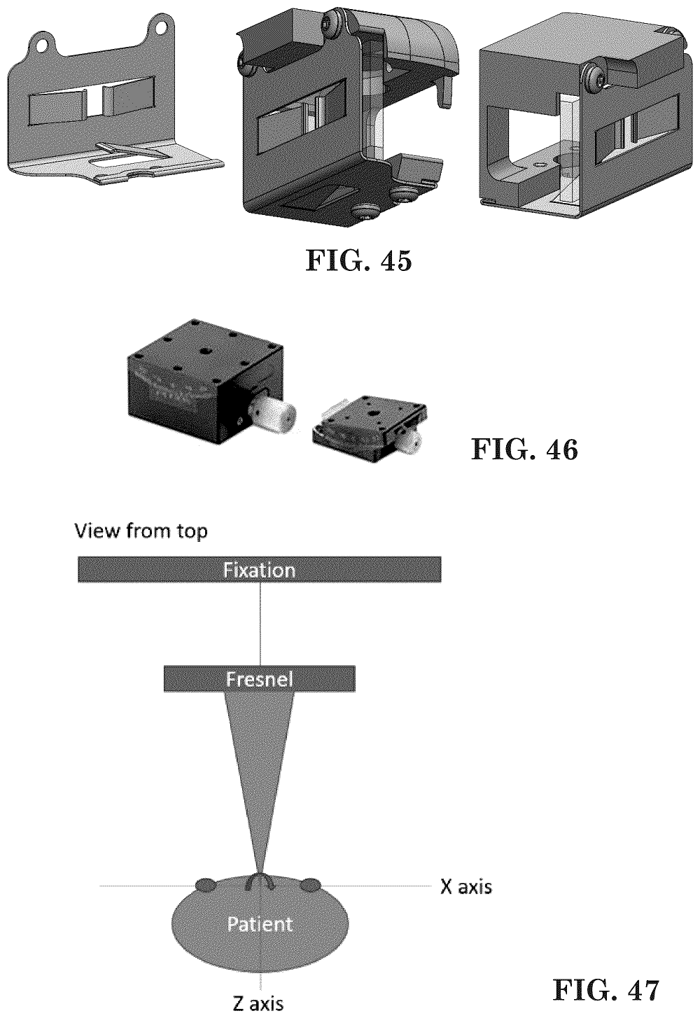

In place of this true kinematic mounting, a preferred approach is to use six point contacts (bumps), three on each of the surfaces of interest. FIG. 43 provides a side view and a bottom view of a preferred mount using six point contacts. FIGS. 44 and 45 provide additional projection and perspective views of the prism mount. The idea is that the prism settles on the bumps evenly on the bumps so that it “wants” to stay there. There are no uneven reaction forces causing the prism to drift to one side or the another.

This mounting scheme is not kinematic because there are extra points of contact that the prism toggles between. Therefore, this only works if the mounting bumps are tightly toleranced both to their theoretical profiles and each other, so that any of the toggle positions are within the acceptable tolerance of the optic.

As seen in the side view, there is a semispherical bump constraining the prism's motion upwards. The reason for the round shape is so the orientation of the prism is only set by the 6 bumps, rather than the untoleranced top surface.

The prism is held against the bumps by a spring clip that pushes the prism into the bumps from the back (right in FIG. 44), and up against the top sphere from the bottom. This is done with cantilever beams made of a high yield strength material to provide a constant force and shock load protection.

This clip hooks around in the front (see FIG. 45) to attach securely to the main mounting piece then screwed in place.

### OCT Office Device/System Concept

In embodiments, the present ophthalmic system may be embodied in an OCT system/device. An exemplary OCT device relies on a custom patient support to align the patient to the optics. This custom patient support is made from a 3D scan of the patient's face. While an approximate pupil location can be gleaned from the 3D scan, there are two major limitations: fixation and accuracy.

Fixation is an issue because even if the pupils could be accurately found from the scan, the patient's pupils will not be aligned in the device unless they had the exact same gaze angle with the scan as in the device. This is highly unlikely because the face scan process involves the scanner and patient moving relative to each other, making it difficult to fixate in one place, let alone the correct place. The present OCT's fixation is straight ahead focused at infinity.

Accuracy is also an issue because in our testing, the 3D scanning app with pupil finding was not able to locate the pupils within the tolerance for the present OCT device. Our target population for the present OCT device is elderly and has small pupils, and the smaller the pupil, the tighter the tolerance to align the optics to the eye in order to get a high quality OCT image.

A present goal is to accurately determine pupil coordinates when the patient is looking at a fixation equivalent to the OCT's relative to a 3D scan of their face. Once this is obtained, the patient support can be built using the pupil location and 3D face geometry data, so that the patient is automatically aligned to their OCT device without a need for adjustments.

A preferred patient support is made from 3D scans of a patient's face/head. The 3D scan may be taken with any 3D scanner known in the art, but is preferably a hand-held 3D scanner, and may be part of a utility provided in some smart phones. The limitation of using a smart phone 3D scanner is that such a scan may have very poor quality in the eye area because eyelashes can obscure the view and the transparent cornea is difficult to scan. There are pupil finding algorithms in the scanning app, but they are only accurate down to around +/−2 mm. This is an issue because a patient pupil may be aligned to the optics within +/−0.5 mm, including several other tolerances.

Due to the accuracy limitations of low-quality 3D scan, in a prototype OCT system, patient testing is done with the OCT optics head mounted to an XYZ stage. In this case, an operator aligns the device to the patient's pupils manually. This cannot be done in the end OCT product because the final OCT product would not have an XYZ stages or any way to fine tune alignment between the patient and the optics. The present system addresses this problem.

Aside from some basic scanning apps with low-accuracy pupil finding, Applicants are not aware of any software or hardware that provides sufficient level of pupil finding and aligning. Usually, when high accuracy is desired, a higher resolution 3D scanner is used. However, these are cost prohibitive, might not be more accurate at scanning the eye area, might not be eye-safe, and would not solve the fixation problem.

The present disclosure provides improved accuracy and correct fixation relative to using a single step scan, even when using a low resolution, handheld 3D scanner, such as a smart phone.

A fundamental concept of the present ophthalmic device is that there are two 3D scanning steps that are used to get both full-face geometry data and accurate pupil locations with fixation. The data from the two steps are then combined, resulting in face scan data that can be used to build a mask with accurate pupil locations. The present approach may be summarized with the following steps:
Step 1 (steps 1 and 2 can be done in either order): using a standalone (e.g., handheld) 3D scanner
   Scan patient's full face (including forehead and chin) with the 3D scanner
Step 2
   Patient sits at office ophthalmic device and looks at a provided fixation point/target.
   The office ophthalmic device adjusts relative to the patient.
   A 3D scanner on (e.g., fixed onto) the office ophthalmic device captures a partial scan of the patient's face
   Cameras on the office ophthalmic device (with known location relative to the 3D scanner on office ophthalmic device) find the X, Y, Z locations of each of the patient's pupils
Step 3—In Software
   Scan from step 2 is related to (e.g., correlated to) scan from step 1 (e.g., using facial fiducial markers/characteristic features), including pupil coordinates.
   Data is outputted to mask making software: scan from step 1 with pupil coordinates from step 2.

A goal of the present approach is to obtain the patient's pupil coordinates relative to the patient's face with the face centered on X=+/−½ IPD, Y=0, Z=0. For ease of discussion, the X axis is defined as the horizontal axis between the pupils, the Y axis is defined as the vertical (gravity) axis, and the Z axis is defined as the optical axis into the pupils. To achieve this, one may use a standalone scanner that can move relative to the patient to capture a full-face scan of the patient including the forehead and chin. In a prototype implementation, a smart phone was used, but another suitable 3D scanner may be used. This scan may not need (or use) fixation, and may not need (or use) pupil finding. The result of this scan is a point cloud with an arbitrary origin. It doesn't matter what the origin is.

The office ophthalmic device (e.g., OCT) provides a patient support, as is known in the art. The patient support provides for a fixed forehead rest with front (Z) support, Canthus mark, and Y-adjustable chin rest with bottom (Y) and front (Z) support (manual but with sensor to measure). This measurement is used to find the bottom and front of the chin to put into the face model—for better accuracy in chin area which is especially helpful for people with fleshy chins or beards.

Everything besides the patient support is mounted on X axis and Y axis goniometers (rotation centered about theoretical patient origin). The patient is not going to be perfectly straight in the patient support, so the device may be rotated to them. FIG. 46 shows exemplary goniometers that may be oriented toward a patient.

The present exemplary approach uses 4 fixed iris cameras: two for each eye. One from the temporal side (to measure pupil location in Y and Z), and another from the bottom (to measure pupil location in X and Z). If the OCT system had an incorporated fundus imager (e.g., fundus camera), the fundus imager's iris cameras may optionally be used. A purpose of measuring from the bottom and the side is to not block the patient's gaze from looking at the fixation ahead of them. These cameras get calibrated relative to the 3D scanner described below so that they are at known locations relative to each other. From the captured images, pupil coordinates relative to the 3D scan on the office ophthalmic device can be determined.

The office ophthalmic device 3D scanner may be characterized by preferably:
Coming from above or below gaze direction to not block the patient's view of the fixation.
Captures a partial scan of the patient's face, from above the chin rest to below the forehead rest.
Outputs a point cloud or 3D mesh file (e.g., PLY, OBJ, or STL file format).
Is calibrated relative to pupil cameras so that they are at known locations relative to each other.

### Fixation

FIG. 47 provides an exemplary application of fixation. The patient looks through a Fresnel lens at a fixation target.

Distances between patient, Fresnel lens, and fixation are preferably set to simulate closely enough fixating at infinity, which is what the typical office ophthalmic device has. The fixation may be blurry for patients with a refractive error (particularly if no refractive error correction is provided), but the fixation is made large and obvious enough that the patient can still fixate even if it is not perfectly clear.

### Workflow

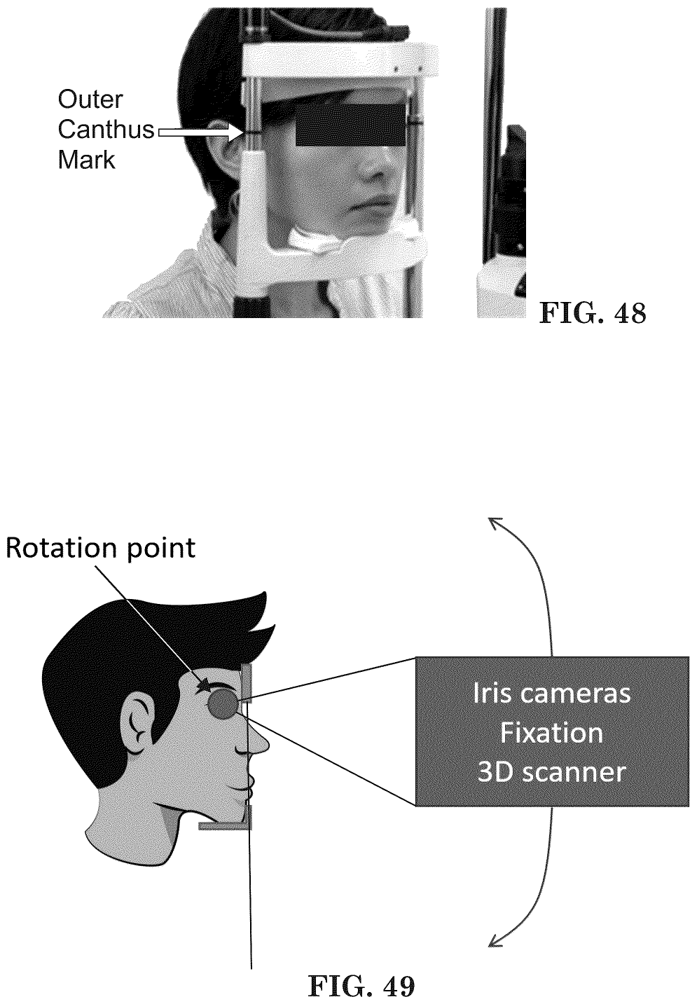

FIG. 48 shows a patient's head positioned within an ophthalmic device's patent support, including outer Canthus marks. The patient sits at the ophthalmic device with their chin all the way forward in the chinrest and forehead against a forehead rest, as is typical of ophthalmic devices, such as an OCT. A technician adjusts chin rest height to put the patient's eyes at the canthus mark.

The technician aligns the patient to the office ophthalmic device (OCT) the best they can and instructs the patient to look at a fixation symbol/spot/point/sprite. As the patient is being adjusted, pupil cameras are tracking the coordinates of the pupils to automate the following processes:
Rotation about X axis:
   A goal is to make sure pupils are fully visible by rotating (e.g., iris cameras/fixation/3D scanner assembly) until eyelid droop is not obscuring view of the pupil. FIG. 49 illustrates rotating an ophthalmic device (e.g., iris cameras/fixation/3D scanner assembly) until an unobstructed view of the pupil is achieved.
   Rotate iris cameras/fixation/3D scanner assembly with X axis goniometer so the patient can fully see the fixation with a comfortable gaze angle.
Rotation about Y axis (evening out Z coordinates):
   A goal is to get OS_Z and OD_Z both to zero (have tolerance of +/−1 mm).
   Rotate iris cameras/fixation/3D scanner assembly with Y axis goniometer to zero out Z coordinates. In various embodiments, this step is included due to the patient support mask having a very limited range of Z values it can accept. The smaller the range, the more compact the mask/slug system, so this step is advantageous to implement.
   A reason to motorize/automate this step is that the patient may not be able to make fine angular adjustments of their head about the Y axis due to age, flexibility, and neck issues. Rotating the device around them ensures that they can stay comfortable and still get measured accurately.
Once instrument is straightened/aligned about the patient and the patient can see the fixation well, capture pupil locations in X, Y, Z and a partial 3D scan is done in relation to those pupil locations.

Once the above steps are complete (both the handheld 3D scan of the patient's face and the office ophthalmic device 3D scan of (at least) the of the eye/pupil area), the data is processed in software.

### Software Processing

Transformations:
   This office device scan's orientations are made the same orientations as one wants to use in the patient's custom mask, so one can translate this model but not rotate it about X or Y axes, as that would have an impact on gaze angle.
   X: translate in X so pupils are at +/−½ IPD
   Y: rotate about Z axis and translate in Y so that Y=0 for both eyes
   Z:
      Should already be very close to 0 for both eyes within a total range of 2 mm. Split the difference and translate in Z so that the Z coordinates are +/−½ Z offset.
      Translate the entire face in Z to offset the face for refractive error offsets in the mask to keep the working distance from the ocular lens to the eye constant when you move the ocular lens. There is an established equation for this.
Relating standalone scanner's face model:
   Import standalone scanner's full face model into office device partial face model
   Use registration algorithm (well established best fit mesh registration algorithm) to align full face scan to office device scan
Export:
   Aligned full face scan with pupil coordinates
   Chin height data

### Mask Multi-Step Manufacturing Processesd

The present exemplary OCT system uses a face mask customized to a patient's face. As discussed above, a 3D cloud of the patient may be generated using a two-step 3D scanning process, and the constructed face mask is used to provide custom head support to the patient. Additive manufacturing has been found to be a cost-effective approach to producing a patient mask, but additive manufacturing currently does not provide sufficient accuracy, particularly for kinematic cone placement in the forehead area, as discussed above. The kinematic cones locate the optics to the patient's pupil, so a misplaced kinematic cone would lead to misalignment of the optics to the patient.

3D scanning, CMM (Coordinate Measuring Machine), and other accurate metrology methods are known in manufacturing to find the quality of finished parts in a quality assurance process to accepted or rejected a finished parts. An example of such metrology software programs is Geomagic Control X™, but it doesn't include features to actively feedback into a manufacturing processes. FormAlloy™ is an additive manufacturing company that actively inspects parts while they are being additively manufactured. However, that data is not used for active feedback during manufacturing.

### Process

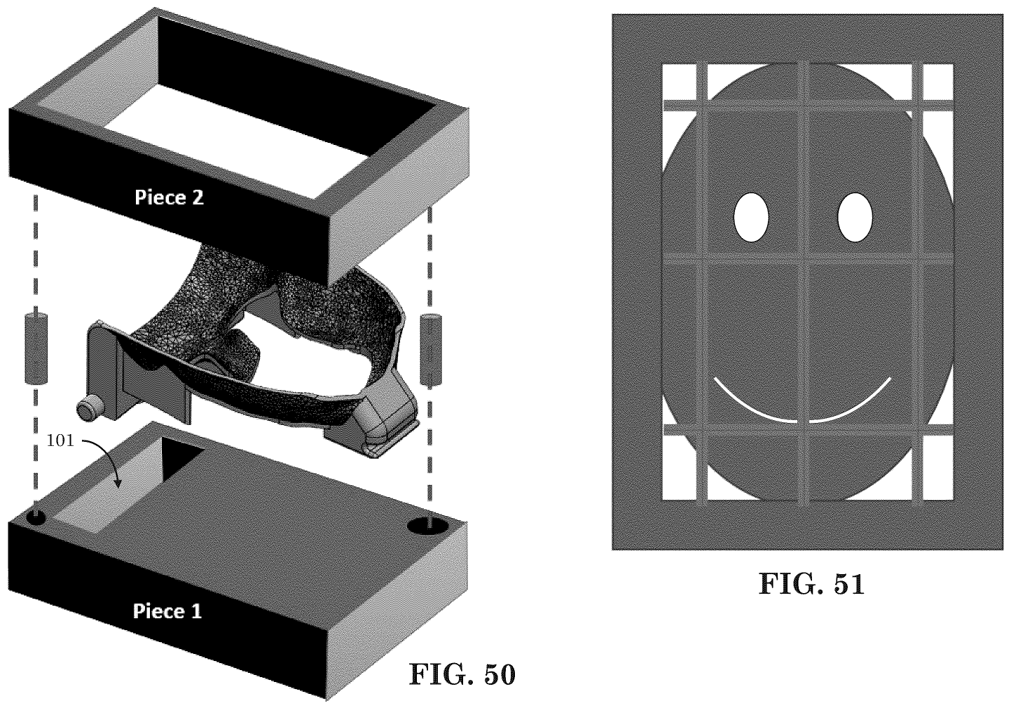

3D print mask: For example, the mask may be printed using HP Multi Jet Fusion™ (MJF) with vapor smoothing. Any suitable printing method would work for this process.
   Features:
      Patient's inverted face surface (mask)
      Approximate locating/clamping features for jig block
      Forehead bosses for cones with extra material to machine away
Clamp mask into jig block. FIG. 50 illustrates a two-piece jig block, in accordance with the various embodiments.
   Accurately machined two-piece block (e.g., Piece 1 and Piece 2)—machined square out of a robust material (i.e. steel) so that the block can be mounted face up or face down and have accurate datums on either side.
   Piece 1 holds and roughly locates the mask with the face surface up
   Piece 2 accurately locates to piece 1 and clamps the mask into the block
   Both parts have openings for the mask—piece 1 has openings 101 in the forehead area for machining cone holes, and piece 2 is open in the face area for measuring the face surface
Measure jig block and mask
   The measuring devices may be a CMM or a 3D scanner. FIG. 51 shows the use of a Coordinate Measuring Machine with the jig of FIG. 50.
   If using a CMM:
      Put jig block in machine, face surface up
      Touch off of edges of the block to find the X/Y/Z/orientation datums
      Measure several points on the mask face surface along a few pre-set X-Y lines:
   If using a 3D scanner
      Scan the entire mask face surface and the top, side, and front surfaces of the jig block
In software, locate mask relative to jig block
   Find “best fit” of CMM points or scan points relative to theoretical mask model
   Relate this located theoretical mask model to the known datums of the jig block
   This will give you an XYZ and angular transformation between the datums and the face surface
Program CNC mill to machine cone holes
   Using the transformed coordinates of where the face surface physically is in the jig block, calculate coordinates of the cone holes relative to jig block datum surfaces
   Result: machine code for cone holes
Put jig block in CNC mill with forehead (piece 1 of jig block) facing upwards
   3 axis mill.
   Reference off of top, side, and front surfaces (datums) of jig block. In various embodiments, this may be done once when setting up the process-hard stops can be set up so that when the jig block is put in again with a different mask, it goes in the exact same place on the machine
   Machine cone holes into forehead of mask. Size should be optimized for press-fit of cones. Hole should have a slight chamfer to help guide the cones in place.
Press in cones
   Keep mask in jig block
   Put jig block in arbor press
   This arbor press setup can be slightly customized to align the jig block and press so that the press head will always be aligned with the cone holes regardless of that patient's exact cone coordinates and the mask is as well-supported as possible to prevent flex during the pressing process
   Place cones into mask forehead—they should easily sit in the right location due to the hole chamfer
   Press cones into mask forehead

### Mask Design Features

As discussed above, the present ophthalmic system uses a 3D printed custom patient support, or mask, to align the patient to the optics. To be fully integrate it into a product, the mask is positioned and mounted to the home device (the present ophthalmic system), which is not adjusted to the patient. The 3D printed mask is used to align the patient to the present ophthalmic system (e.g., OCT). To be viable in mass production, the masks should be designed in a matter of minutes, not hours or days.

Herein is presented an equation-driven design for eye opening, face length, cone locations, and refractive error. The starting point for each mask is a template that contains all of the common dimensions and mounting features (more on this in the automated section). However, some of the features cannot be kept common for all faces, such as eye opening width, mask vertical length, cone locations, and refractive error. A goal for eye opening width and mask vertical length is to maximize the amount of surface contact between the patient and the mask while making sure there isn't mask material covering their eyes or mouth.

### Eye Opening Width

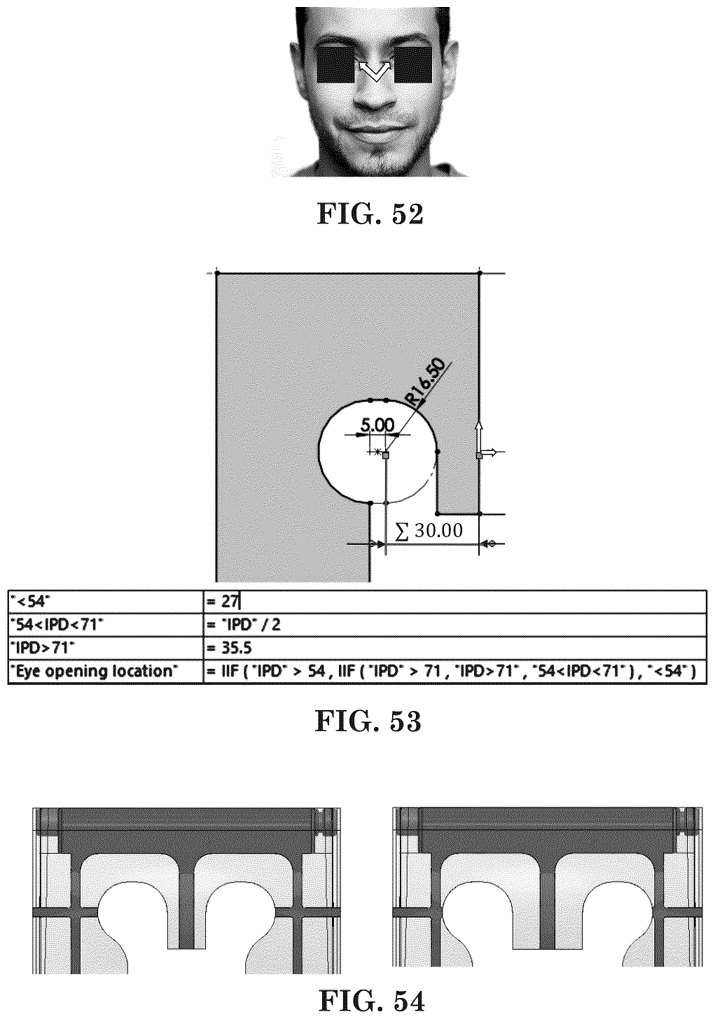

FIG. 52 shows using the nose (particularly the nose bridge) as a reference for determining the eye opening width. The nose is a very helpful landmark for aligning the patient horizontally because it is one of the few “hard points” on the face. The more contact the nose piece has with the sides of the patient's nose bridge, the better it can locate the face. For eye opening width, the idea is to make the nose piece as wide as possible without going into the inner corner of the patient's eye:

FIG. 53 shows a method for making the nose piece as wide as possible without going into the inner corner of the patient's eye. This is done by adjusting the position of the eye opening (blue highlighted dimension) according to the patient's interpupillary distance (IPD): Left: eye opening for a patient with a 50 mm IPD and Right: eye opening for a patient with a 75 mm IPD. FIG. 54 show an example of a resultant nose pieces on a face mask (e.g., front view and back view).

### Face Length

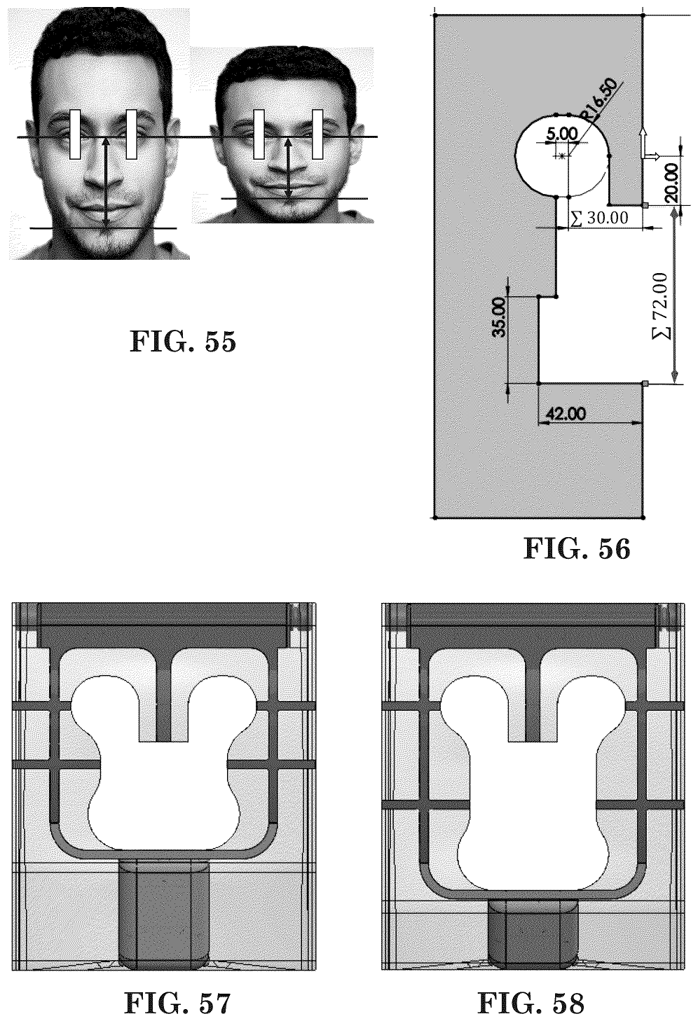

The same goals apply with the face length—it is desirable for as much of the front of the chin to be supported, but the mouth should not be covered. Since people have widely ranging face lengths, the distance is also adjusted. FIG. 55 illustrates the adjusting of face length. This adjustment happens in a way that doesn't interfere with the common mounting at the top and bottom of the mask. As a result, the mask template is designed around the longest face, with the face opening brought in for shorter faces.

FIG. 56 shows the adjusting of size between long and short faces. The blue dimension is what is adjusted. FIGS. 57 and 58 show the result adjusting for face length. For any face length, the mask internals are moved accordingly, but the exterior mounting features remain the same.

### Kinematic Cone Locations

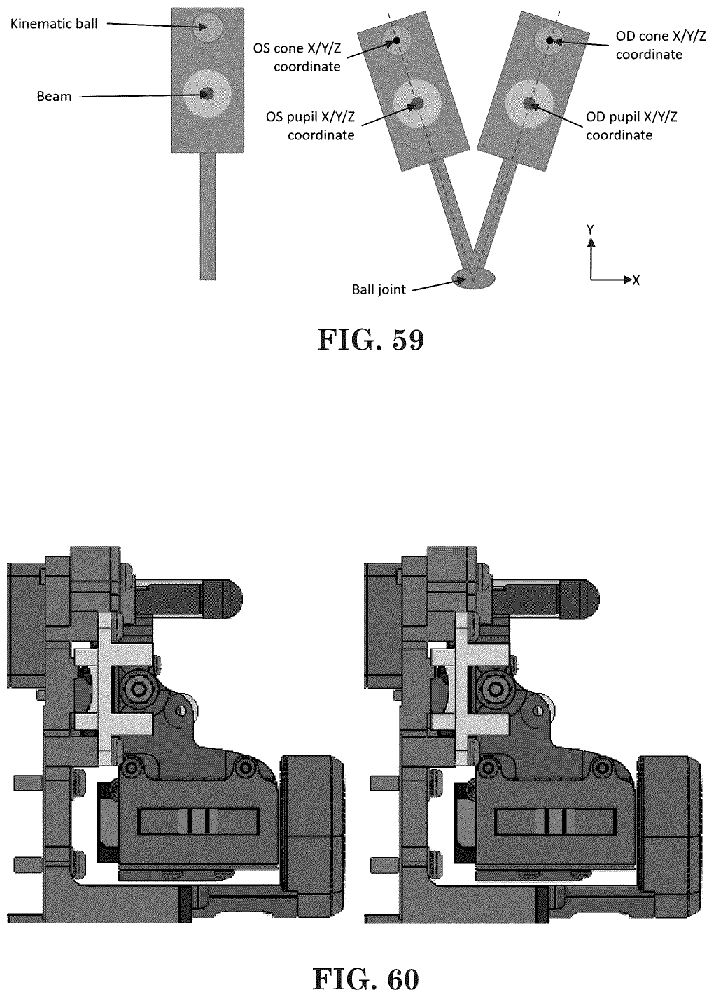

The next adjustment is the kinematic cone locations. FIG. 59 shows a method of determining kinematic cone locations for a mask. Because of the polar/pogo coordinate system and the geometry of the device with the kinematic ball offset from the beam, the cone coordinates are not simply the patient's IPD with a different Y value.

Ideally, the patient's pupil coordinates would simply by X=+/−½ IPD, Y=0, Z=0 and this calculation would be simple, just accounting for lever arms and the angle of the optics head. However, there are anticipated (office ophthalmic device errors) and yet unidentified reasons that the pupil coordinates could have non-zero values in Y and Z.

Input X/Y/Z pupil coordinates may be mapped to corresponding kinematic cone center coordinates. These cone coordinates can be used when designing the mask to build bosses and cut out material to house the cones, which are pressed in after printing. Knowing where the cones are going before printing the mask is helpful to locate supporting structures in the print, minimizing the amount of post-processing that may be done after printing.

### Refractive Error Offset

FIG. 60 shows the adjusting of an ophthalmic device for different refractive errors, in accordance with the various embodiments. The present ophthalmic system/device (OCT) is adjusted for different refractive errors by moving the ocular lens in and out of the device, while leaving the rest of the optics and the kinematic ball in the same place.

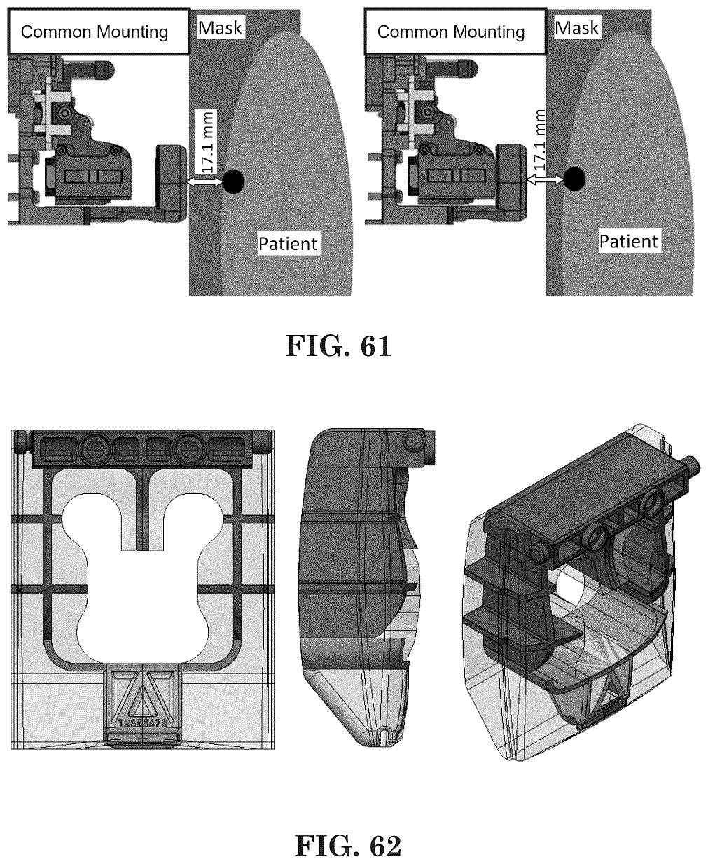

However, one may maintain a working distance between the ocular lens apex and patient's corneal apex of 17.1 mm. If this ocular lens moves in and out relative to the device, then the patient should also move in and out relative to the device. Luckily, the refractive error is known before the mask is created, so one can account for it by offsetting the patient's face model relative to the mask origin by the same amount and direction as the ocular lens is moving on their device. FIG. 61 shows how the mask thickness can be made to compensate for positioning of the ocular lens due to a patient's refractive error to maintain a constant working distance (e.g., 17.1 mm) between the ocular lens apex and patient's corneal apex.

### Automated Mask Generation Process

To automate mask design, one may start with a template. The template consists of 3 portions. FIG. 62 highlights the three main parts/portion of a mask template, in accordance with the various embodiments.
Main mask body—shown in translucent grey in FIG. 62. This body contains the rear (towards the patient) mask shape and goes all the way forward to the device's face plate to allow as many patients as possible to fit on the device.
Mounting features bodies—shown in red and yellow in FIG. 62. These bodies contain the interfaces with the ophthalmic device for locating and attaching the mask.
Stiffening ribs body—shown in green. This body contains a bridge between the top and bottom mounting features to have continuous stiffening features the full length of the mask.

Note: the present system has a main mask body, mounting features bodies, and stiffening ribs.

Next in the automation process is importing and applying the parameters described above: eye opening, face length, cone locations, and refractive error offset. One more parameter that is imported is the Patient ID, which is turned into an embossed label on the mask. All of these parameters are recorded data in the face scan app (possibly combined with the office device), so automating them is straightforward.

Finally, the patient's face model, also from the face scan app is brought in. The origin of the face model in the face app is set to be the center of the two eyes, which is the same as the origin on the mask template. Therefore, when the face model is brought in at the mask's origin, it is in the correct location.

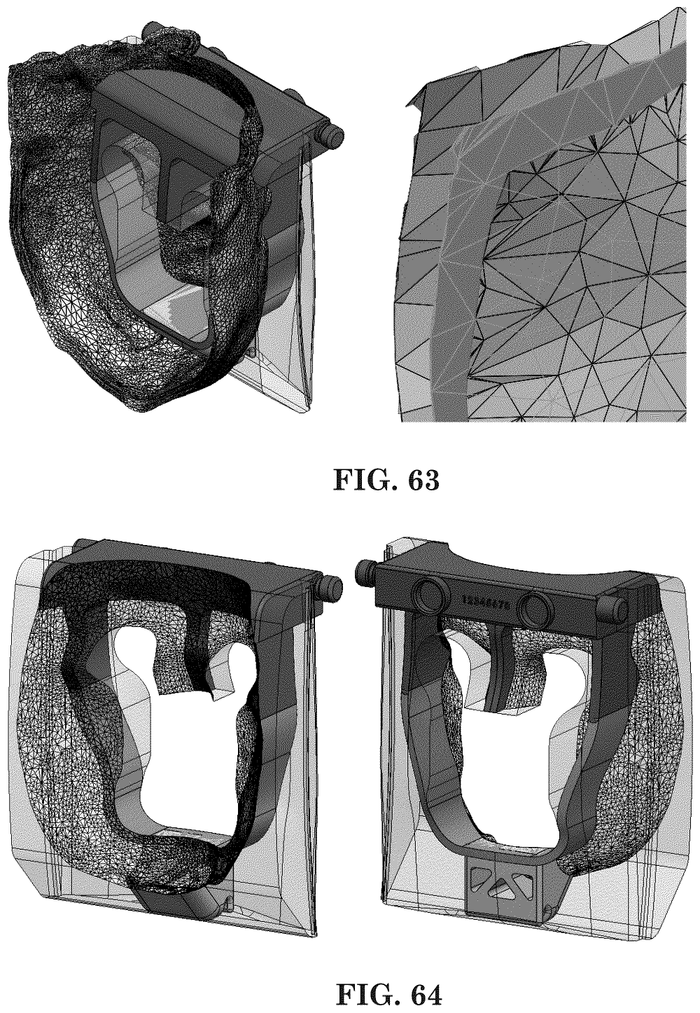

FIGS. 63-67 show various stages in the construction of custom patient face mask, including some front and back view pairs. The face model is offset twice in the direction away from the patient. The first offset is at the desired global thickness of the mask (e.g., using 3 mm), and the second offset is at the desired rib thickness (e.g., using 6 mm from the patient's face surface).

First Boolean subtraction: subtract from all bodies the material on the patient side of the nominal patient surface. This creates the patient interface (see FIG. 64).

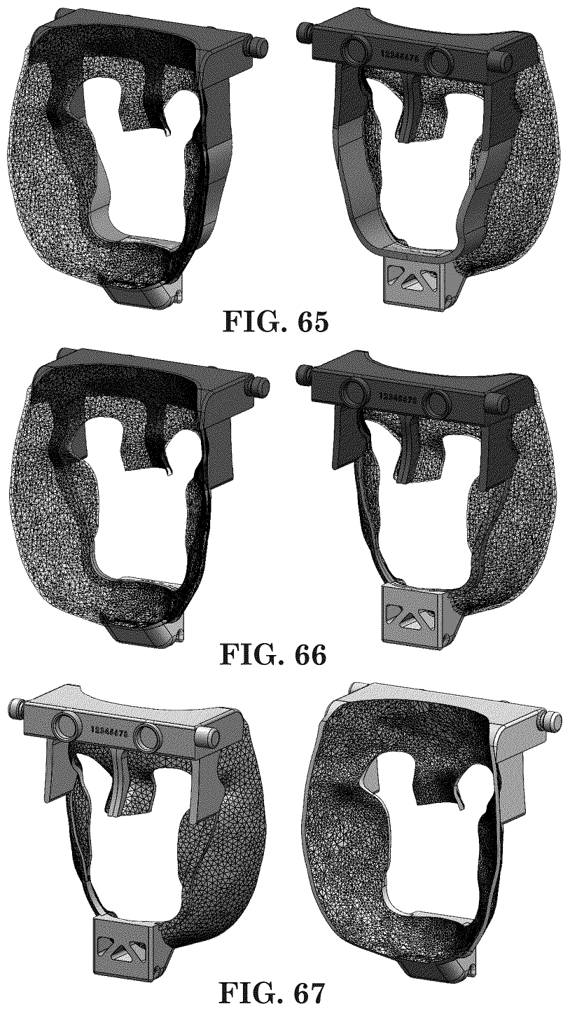

Second Boolean subtraction: subtract from the main mask body the material on the Kepler device side of the mask thickness (3 mm) surface. This sets the global mask thickness (see FIG. 65).

Third Boolean subtraction: subtract from the stiffening ribs body the material on the Kepler device side of the rib thickness (6 mm) surface. This sets the rib thickness (see FIG. 66).

Combine bodies: combine the main mask body, mounting features bodies, and stiffening ribs bodies so that the mask is all one solid body (see FIG. 67).

These are all Boolean operations that work regardless of the patient's face shape. These processes are therefore also straightforward to automate because the processes may not need any inputs beyond importing the patient's 3d face model.

### Mask Mounting Handlebar

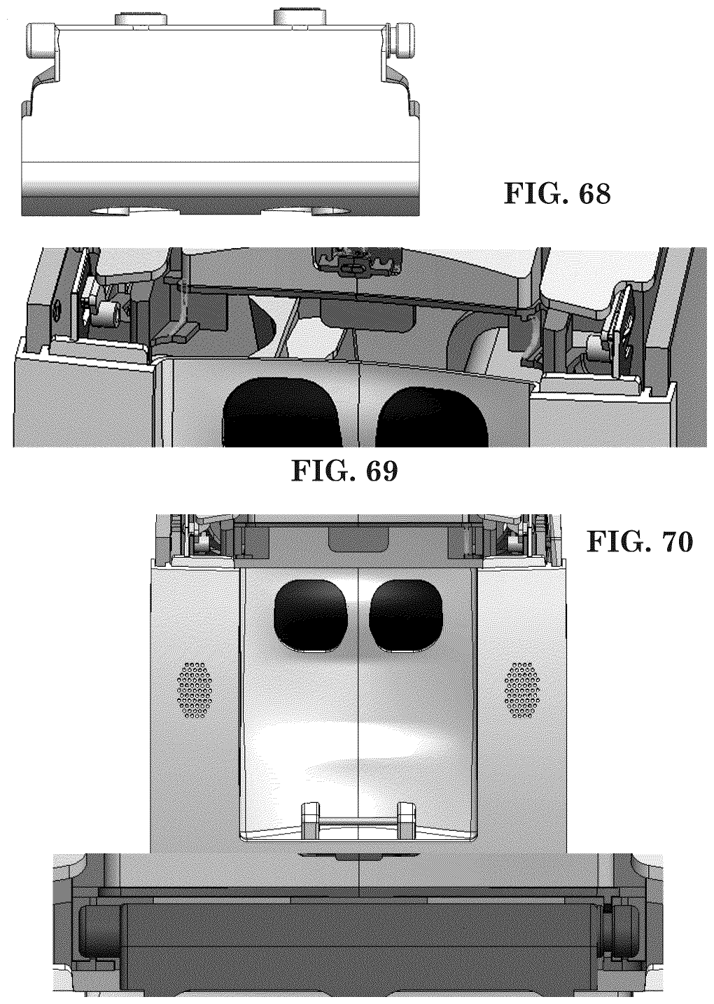

The mask is mounted to the ophthalmic device with two features-the handlebar and the chin bar. FIG. 68 is an example of the handlebar feature, shown in the template from the top, and consisting of a horizontal bar protruding from each side of the forehead portion of the mask. The OD side of the handlebar has a small notch cut into it.

The mask is placed into the ophthalmic device (e.g., OCT of fundus imager/camera) with the top cover removed. FIG. 69 shows the placement of the mask onto the ophthalmic device. The handlebar fits into the purple piece, and is constrained from moving downwards or further into the device by the blue highlighted surfaces.

FIG. 70 shows a blue highlighted surface that fits inside the notch, constraining the mask from moving.

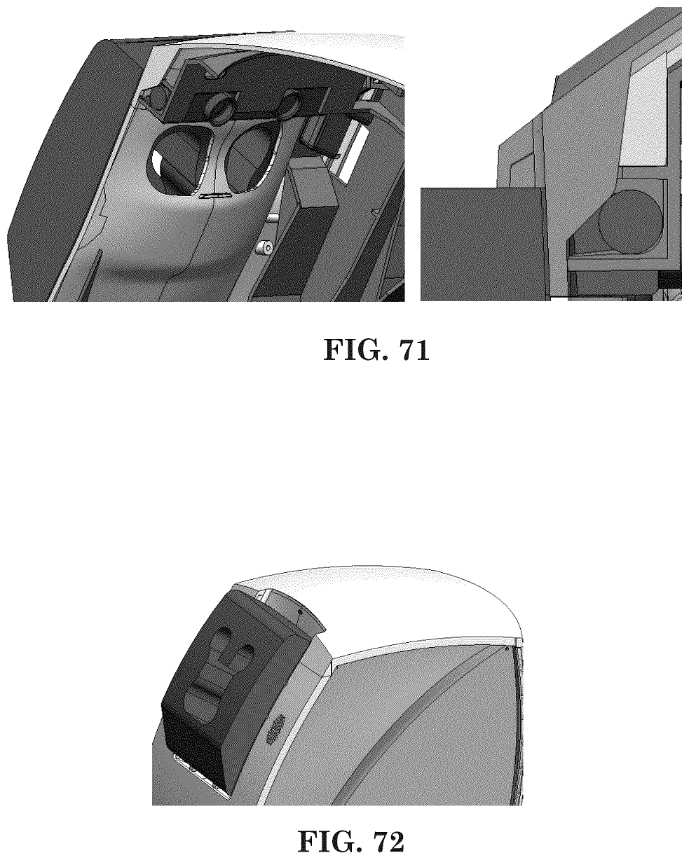

FIGS. 71 and 72 show the final steps in coupling the face mask to the ophthalmic device. Once the mask is in place (as well as the smile), the top cover is installed. This secures the handlebar into the device by clamping on the top and patient side of the mask, pushing the handlebar into the purple structural piece.

This handlebar design is not intended to provide super accurate alignment. Rather, it is intended to be an easy way to roughly align the mask to the device and hold it in securely. The fine alignment occurs between the kinematic ball and the cones, which are somewhat isolated from the initial placement of the mask.

### Mask Mounting Chin Hook

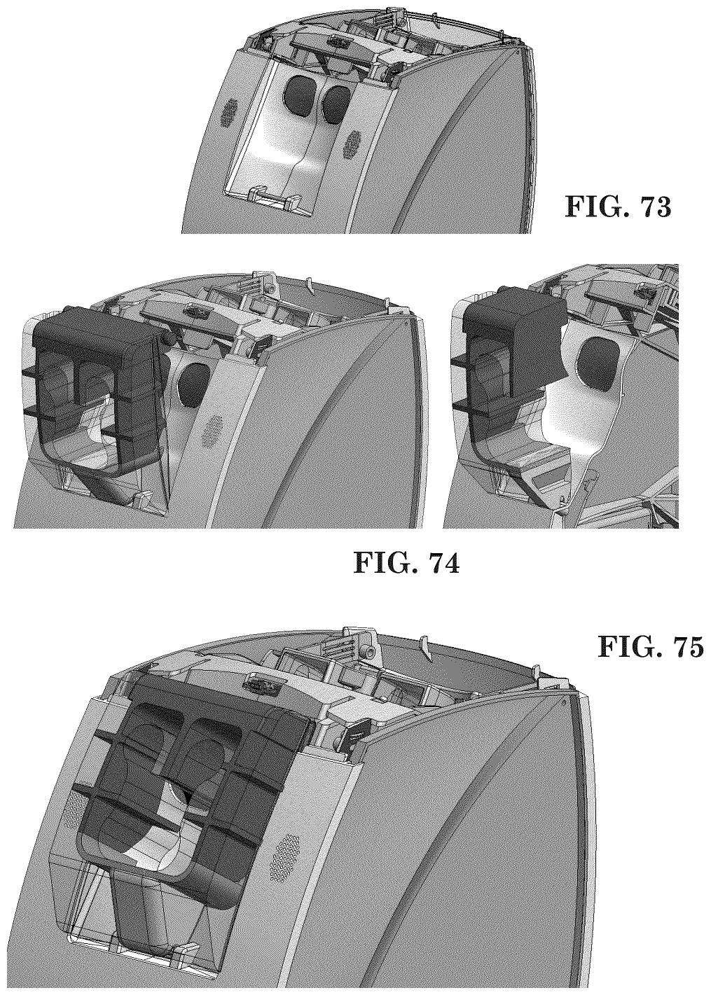

FIGS. 73-76 illustrate the process of mounting the face mask to an ophthalmic device. The process begins with removing its cover (see FIG. 73).

Then the mask is hooked on at the bottom by putting the upside-down U-shaped opening onto the chin bar (FIG. 74).

Then the mask is pivoted into the device from the bottom until the handlebar is in place, and the rest of the process from the Mask Mounted Handlebar section, above, is followed to lock the mask in place (FIG. 75).

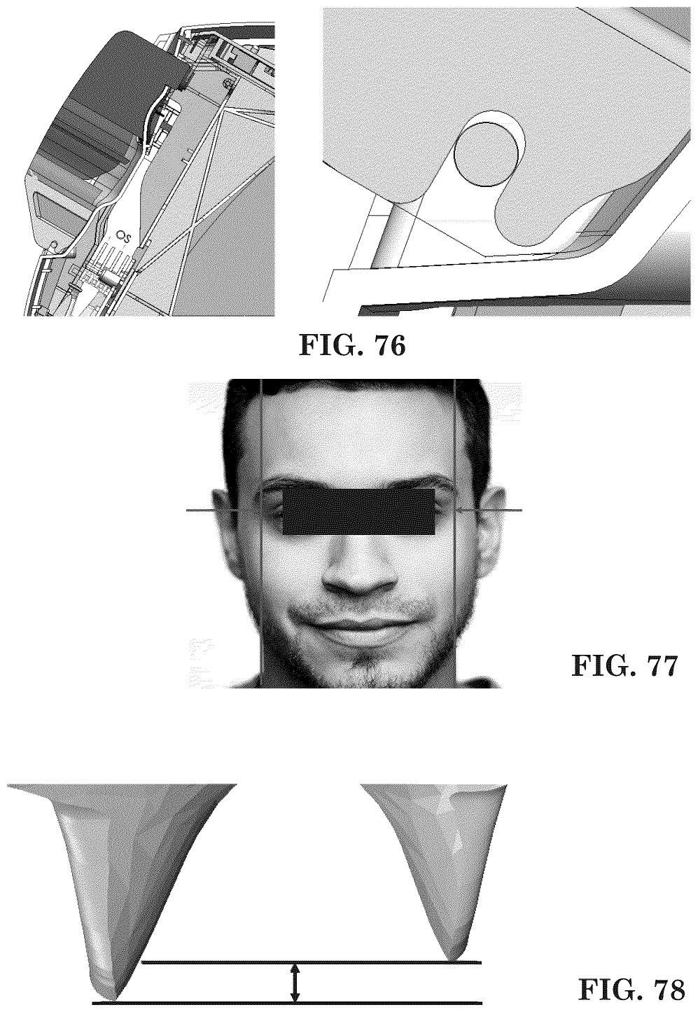

One feature to note: when the handlebar is locked in place, the mask lifts off slightly from the chin pin in the vertical direction. This is to prevent the chin bar from over-constraining the mask. However, it still provides support for the mask from rotating counterclockwise, as shown in FIG. 76, when the patient leans into it.

### Side Compression

The present (OCT) system may include the tightest alignment between the patient and the optics in the X direction (left-right). However, skin/flesh on the face are inherently compliant, and thus not great alignment datums. To overcome this compliance, the mask should preferably be as tight as possible to the patient's face without being excessively uncomfortable. “Excessively uncomfortable” here means that the mask is so tight that the patient's alignment worsens because they can't put their face in the correct position or hold it there for the duration of an ophthalmic (OCT/fundus imaging) scan.

A 10% compression in the X direction of the temple portion (beyond X=+/−55 mm) of the mask is sufficient. This means that the center area of the face is uncompressed, and then each side is compressed to 90% of its delta from the +/−55 mm plane. FIG. 77 shows this partial compression of the face to compensate for a patient's face being compliant (tissue is malleable).

### Centering Nosepiece Around the Nose

It is noted that faces are not particular symmetrical. With the scheme described above, the mask is centered around the location of the eyes, without regard for the nose. FIG. 78 is an example of a patient with an off-center nose resulting in an uneven nosepiece on the mask, as seen here from the top.

Uneven nosepieces, where the left or right side of the nose is more supported than the other, may result in poor X location of the patient. One potential solution is to keep the mask mounting centered around the patient's eyes (so their pupil coordinates are still +/−½ IPD), but shift the nose piece so that it is centered on the patient's nose. There are limits as to how far the nosepiece can move before it runs into ocular lens exclusion zones, but this should at least improve the left-right nose support evenness.

1. A system for course alignment of an ophthalmic device, comprising:
a set of internal moving parts of a device structure;
a dorsal pin coupled to a dorsal fin; and
a pogo pin,
wherein the dorsal pin is inserted through a proximal part of the dorsal fin,
wherein the dorsal fin is further configured to freely slide in a Y-Z plane within the set of internal moving parts,
wherein the pogo pin is configured to slide vertically in a Y-direction within the set of internal moving parts along a Y-axis, and
wherein the pogo pin is seated within a spherical bearing.

2. The system of claim 1, wherein the insertion of the dorsal pin through the proximal part of the dorsal fin to constrain angular rotation of the set of internal moving parts of the device structure.

3. The system of claim 1, wherein the spherical bearing that constrains motion of the pogo pin in both an X and Z direction.

4. The system of claim 1, wherein the angular rotation of the set of internal moving parts is configured around the Y-axis by the dorsal pin.

5. The system of claim 2, wherein the dorsal fin is supported and attached on both ends to a stationary structural part of the device structure.

6. The system of claim 5 wherein the attachment to the stationary structural part of the device structure enables the dorsal fin to pivot on the dorsal pin while the dorsal pin is confined within the set of internal moving parts.

7. The system of claim 5, wherein the Y-axis is configured with an adjustable angle in accordance with a position and angle of an optics head located within the set of internal moving parts.

8. The system of claim 2, further comprising a pair of extension springs between the set of internal moving parts and the dorsal fin, wherein the pair of extension springs are configured to absorb or lessen impacts on the set of internal moving parts of the device structure.

9. The system of claim 2, further comprising an assembly of the dorsal fin with an inserted dorsal pin, and the pogo pin seated in the spherical bearing.

10. The system of claim 9, wherein the assembly is axially configured in a Z-axis direction towards a patient's eye.

11. The system of claim 10, wherein the assembly is configured to move within a range of motion comprising a cone shape with translation in at least one of an upward direction or a downward direction.

12. A system for guidance of an ophthalmic device comprising:
a U-shape part; and
an optics head,
wherein the U-shape part comprises a track configured in a U-shape pathway,
wherein the optics head of the ophthalmic device is guided in a U-shape pattern via the U-shape part.

13. The system of claim 12, wherein the U-shape pattern enables the optics head to circumvent a protrusion caused by a nose on a face, while traversing between a right eye and a left eye of the face.

14. The system of claim 13, wherein the U-shape part enables positioning of the optics head within a range of a pair of kinematics cones corresponding to the right eye and to the left eye.

15. The system of claim 14, wherein the U-shape part enables positioning of a kinematic ball of the ophthalmic device for acceptance to a corresponding kinematic cone.

16. The system of claim 15, wherein the U-shape part is configured with a width that conforms to an interpupillary distance (IPD) between a left eye and a right eye of the face.

17. An interface comprising a gearbox disposed in an ophthalmic device, wherein the gearbox comprises:
a worm wheel coupled to a worm gear;
a pin plate;
a bump; and
an optical head,
wherein the pin plate is rested inside the worm wheel,
wherein the worm wheel is configured to be rotated by a pair of torsion springs in accordance with a movement of the optical head via a U-shaped pathway across a patient's face, and
wherein the worm wheel is configured to compress the bump to drive the pin plate.

18. The interface of claim 17, wherein the gearbox further comprises:
a set of internal moving parts;
a kinematic cone; and
a kinematic ball,
wherein the set of internal moving parts is prevented from further movement in response to contact of the kinematic cone by the kinematic ball of the ophthalmic device.

19. The interface of claim 18, wherein the gearbox further comprises a DC motor configured to compress a torsion spring in the gearbox until a current threshold is met for the DC motor, and wherein an overdrive operation of the torsion spring by the DC motor is performed to properly seat the kinematic ball in the kinematic cone prior to stoppage of the DC motor.

20. The interface of claim 19, wherein in response to the current threshold being exceeded, the current threshold can act as a safety switch for stoppage of the DC motor.

21. The interface of claim 20, wherein in response to the current threshold being exceeded prior to a completion by the worm wheel of a movement of the optical head in a U-shape pathway, the current threshold provides notice of a fault in the gearbox.

22. The interface of claim 21, wherein a large ratio of the worm gear to the worm wheel allows a low-powered DC motor to generate sufficient torque for movement of the optical head in the U-shape pathway.

23. The interface of claim 22, wherein the worm gear prevents a back-driven operation of the optical head, in response to the DC motor being powered down.
